(19) United States
(12) Patent Application Publication
(10) Pub. No.: US 2020/0270237 A1
(43) Pub. Date: Aug. 27, 2020

(54) NEW ALKOXYAMINO COMPOUNDS FOR TREATING PAIN AND PAIN RELATED CONDITIONS

(71) Applicant: ESTEVE PHARMACEUTICALS, S.A., Barcelona (ES)

(72) Inventors: Carmen ALMANSA-ROSALES, Barcelona (ES); Félix CUEVAS-CORDOBES, Valdemoro (ES)

(21) Appl. No.: 16/649,725

(22) PCT Filed: Oct. 19, 2018
(86) PCT No.: PCT/EP2018/078708

§ 371 Date: Mar. 23, 2020

(30) Foreign Application Priority Data
Oct. 19, 2017 (EP) 17382698.3

(51) Int. Cl. C07D 409/14; C07D 409/12; C07D 211/16; C07D 241/04; C07D 295/195; C07D 405/04; C07D 231/12; C07D 487/04

(52) U.S. Cl. CPC C07D 409/14; C07D 409/12; C07D 211/16; C07D 487/04; C07D 295/195; C07D 405/04; C07D 231/12; C07D 241/04

(57) ABSTRACT

The present invention relates to new compounds of general formula (I) showing great affinity and activity towards the subunit α2δ of voltage-gated calcium channels (VGCC), especially the α2δ-1 subunit of voltage-gated calcium channels or dual activity towards the subunit α2δ of voltage-gated calcium channels (VGCC), especially the α2δ-1 subunit of voltage-gated calcium channels, and the μ-opioid receptor (MOR or mu-opioid receptor). The invention is also related to the process for the preparation of said compounds as well as to compositions comprising them, and to their use as medicaments.

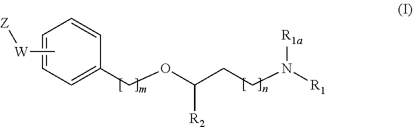

### FIELD OF THE INVENTION

The present invention relates to new compounds that show great affinity and activity towards the subunit α2δ of voltage-gated calcium channels (VGCC), especially the α2δ-1 subunit of voltage-gated calcium channels or dual activity towards the subunit α2δ of voltage-gated calcium channels (VGCC), especially the α2δ-1 subunit of voltage-gated calcium channels, and the μ-opioid receptor (MOR or mu-opioid receptor). The invention is also related to the process for the preparation of said compounds as well as to compositions comprising them, and to their use as medicaments.

### BACKGROUND OF THE INVENTION

The adequate management of pain represents an important challenge, since currently available treatments provide in many cases only modest improvements, leaving many patients unrelieved (Turk, D. C., Wilson, H. D., Cahana, A.; 2011; Lancet, 377; 2226-2235). Pain affects a big portion of the population with an estimated prevalence of 20% and its incidence, particularly in the case of chronic pain, is increasing due to the population ageing. Additionally, pain is clearly correlated to comorbidities, such as depression, anxiety and insomnia, which leads to important productivity losses and socio-economical burden (Goldberg, D. S., McGee, S. J.; 2011; BMC Public Health 11; 770). Existing pain therapies include non-steroidal anti-inflammatory drugs (NSAIDs), opioid agonists, calcium channel blockers and antidepressants, but they are much less than optimal regarding their safety ratio. All of them show limited efficacy and a range of secondary effects that preclude their use, especially in chronic settings.

Voltage-gated calcium channels (VGCC) are required for many key functions in the body. Different subtypes of voltage-gated calcium channels have been described (Zamponi at al.; Pharmacol. Rev.; 2015; 67; 821-870). The VGCC are assembled through interactions of different subunits, namely α1 (Ca_{V}α1), β (Ca_{V}β) α2δ (Ca_{V}α2δ) and γ (Ca_{c}γ). The α1 subunits are the key porous forming units of the channel complex, being responsible for Ca²⁺ conduction and generation of Ca²⁺ influx. The α2δ, β, and γ subunits are auxiliary, although they are very important for the regulation of the channel since they increase the expression of α1 subunits in the plasma membrane as well as modulate their function resulting in functional diversity in different cell types. Based on their physiological and pharmacological properties, VGCC can be subdivided into low voltage-activated T-type (Caᵥ3.1, Caᵥ3.2, and Caᵥ3.3), and high voltage-activated L-(Caᵥ1.1 through Caᵥ1.4), N-(Caᵥ2.2), P/Q-(Caᵥ2.1), and R-(Caᵥ2.3) types, depending on the channel forming Caᵥα subunits. All of these five subclasses are found in the central and peripheral nervous systems. Regulation of intracellular calcium through activation of these VGCC plays obligatory roles in: 1) neurotransmitter release, 2) membrane depolarization and hyperpolarization, 3) enzyme activation and inactivation, and 4) gene regulation (Perret and Luo; Neurotherapeutics; 2009; 6; 679-692; Zampon at al., 2015; Neumaier et al.; Prog. Neurobiol.; 2015; 129; 1-36). A large body of data has clearly indicated that VGCC are implicated in mediating various disease states including pain processing. Drugs interacting with the different calcium channel subtypes and subunits have been developed. Current therapeutic agents include drugs targeting L-type Ca_{V}1.2 calcium channels, particularly 1,4-dihydropyridines, which are widely used in the treatment of hypertension. T-type (Ca_{V}3) channels are the target of ethosuximide, widely used in absence epilepsy. Ziconotide, a peptide blocker of N-type (Ca_{V}2.2) calcium channels, has been approved as a treatment of intractable pain.

The Caᵥ1 and Caᵥ2 subfamilies contain an auxiliary α2δ subunit which is the therapeutic target of the gabapentinoid drugs of value in certain epilepsies and chronic neuropathic pain (Perret and Luo, 2009; Vink and Alewood; British J. Pharmacol.; 2012; 167; 970-989). To date, there are four known α2δ subunits, each encoded by a unique gene and all possessing splice variants. Each α2δ protein is encoded by a single messenger RNA and is post-translationally cleaved and then linked by disulfide bonds. Four genes encoding α2δ subunits have now been cloned, α2δ-1 was initially cloned from skeletal muscle and shows a fairly ubiquitous distribution. The α2δ-2 and α2δ-3 subunits were subsequently cloned from brain. The most recently identified subunit, α2δ-4, is largely non-neuronal. The human α2δ-4 protein sequence shares 30, 32 and 61% identity with the human α2δ-1, α2δ-2 and α2δ-3 subunits, respectively. The gene structure of all α2δ subunits is similar. All α2f subunits show several splice variants (Davies et al.; Trends Pharmacol. Sci.; 2007; 28; 220-228; Dolphin, A. C.; Nat. Rev. Neurosci.; 2012; 13; 542-555; Dolphin, A. C.; Biochim. Biophys. Acta; 2013; 1828; 1541-1549).

The Caᵥα2δ-1 subunit may play an important role in neuropathic pain development (Perret and Luo, 2009; Vink and Alewood, 2012). Biochemical data have indicated a significant Caᵥα2δ-1, but not Caᵥα2δ-2, subunit upregulation in the spinal dorsal horn, and DRG (dorsal root ganglia) after nerve injury that correlates with neuropathic pain development. In addition, blocking axonal transport of injury-induced DRG Caᵥα₂δ-1 subunit to the central presynaptic terminals diminishes tactile allodynia n nerve injured animals, suggesting that elevated DRG Caᵥα2δ-1 subunit contributes to neuropathic allodynia.

The Caᵥα2δ-1 subunit (and the Caᵥα2δ-2, but not Caᵥα2δ-3 and Caᵥα2δ-4, subunits) is the binding site for gabapentin which has anti-allodynic/hyperalgesic properties in patients and animal models. Because injury-induced Caᵥα2δ-1 expression correlates with neuropathic pain, development and maintenance, and various calcium channels are known to contribute to spinal synaptic neurotransmission and DRG neuron excitability, injury-induced Caᵥα2δ-1 subunit upregulation may contribute to the initiation and maintenance of neuropathic pain by altering the properties and/or distribution of VGCC in the subpopulation of DRG neurons and their central terminals, therefore modulating excitability and/or synaptic neuroplasticity in the dorsal horn. Intrathecal antisense oligonucleotides against the Caᵥα2δ-1 subunit can block the nerve injury-induced Caᵥα2δ-1 upregulation and prevent the onset of allodynia and reserve established allodynia.

As above mentioned, the α2δ subunits of VGCC form the binding site for gabapentin and pregabalin which are structural derivatives of the inhibitory neurotransmitter GABA although they do not bind to GABAA, GABAB, or benzodiazepine receptors, or alter GABA regulation in animal brain preparations. The binding of gabapentin and pregabalin to the Caᵥα2δ-1 subunit results in a reduction in the calcium-dependent release of multiple neurotransmitters, leading to efficacy and tolerability for neuropathic pain management. Gabapentinoids may also reduce excitability by inhibiting synaptogenesis (Perret and Luo, 2009; Vink and Alewood, 2012, Zamponi at al., 2015).

Thus, the present invention relates to compounds with inhibitory effect towards the α2δ subunits of voltage-gated calcium channels, preferably towards the α2δ-1 subunit of voltage-gated calcium channels.

As mentioned before, there are few available therapeutic classes for the treatment of pain, and opioids are among the most effective, especially when addressing severe pain states. They act through three different types of opioid receptors (mu, kappa and gamma) which are transmembrane G-protein coupled receptors (GPCRs). Still, the main analgesic action is attributed to the activation of the μ-opioid receptor (MOR). However, the general administration of MOR agonists is limited due to their important side effects, such as constipation, respiratory depression, tolerance, emesis and physical dependence [Meldrum, M. L. (Ed.). Opioids and Pain Relief: A Historical Perspective. Progress in Pain Research and Management, Vol 25. IASP Press, Seattle, 2003]. Additionally, MOR agonists are not optimal for the treatment of chronic pain as indicated by the diminished effectiveness of morphine against chronic pain conditions. This is especially proven for the chronic pain conditions of neuropathic or Inflammatory origin, in comparison to its high potency against acute pain. The finding that chronic pain can lead to MOR down-regulation may offer a molecular basis for the relative lack of efficacy of morphine in long-term treatment settings [Dickenson, A. H., Suzuki, R. Opioids in neuropathic pain: Clues from animal studies. Eur J Pain 9, 113-6 (2005)]. Moreover, prolonged treatment with morphine may result in tolerance to its analgesic effects, most likely due to treatment-induced MOR down-regulation, internalization and other regulatory mechanisms. As a consequence, long-term treatment can result in substantial increases in dosing in order to maintain a clinically satisfactory pain relief, but the narrow therapeutic window of MOR agonists finally results in unacceptable side effects and poor patient compliance.

Polypharmacology is a phenomenon in which a drug binds multiple rather than a single target with significant affinity. The effect of polypharmacology on therapy can be positive (effective therapy) and/or negative (side effects). Positive and/or negative effects can be caused by binding to the same or different subsets of targets; binding to some targets may have no effect. Multi-component drugs or multi-targeting drugs can overcome toxicity and other side effects associated with high doses of single drugs by countering biological compensation, allowing reduced dosage of each compound or accessing context-specific multitarget mechanisms. Because multitarget mechanisms require their targets to be available for coordinated action, one would expect synergies to occur in a narrower range of cellular phenotypes given differential expression of the drug targets than would the activities of single agents. In fact, it has been experimentally demonstrated that synergistic drug combinations are generally more specific to particular cellular contexts than are single agent activities, such selectivity is achieved through differential expression of the drugs' targets in cell types associated with therapeutic, but not toxic, effects (Lehar et al.; Nat. Biotechnol.; 2009; 27; 659-666).

In the case of chronic pain, which is a multifactoral disease, multi-targeting drugs may produce concerted pharmacological intervention of multiple targets and signaling pathways that drive pain. Because they actually make use of biological complexity, multi-targeting (or multi-component drugs) approaches are among the most promising avenues toward treating multifactoral diseases such as pain (Gilron et al.; Lancet Neurol.; 2013; 12(11); 1084-1095). In fact, positive synergistic interaction for several compounds, including analgesics, has been described (Schröder et al; J. Pharmacol. Exp. Ther.; 2011; 337; 312-320; Zhang at al.; Cell Death Dis.; 2014; 5; e1138; Gilron et al., 2013).

Given the significant differences in pharmacokinetics, metabolisms and bioavailability, reformulation of drug combinations (multi-component drugs) is challenging. Further, two drugs that are generally safe when dosed individually cannot be assumed to be safe in combination. In addition to the possibility of adverse drug-drug interactions, if the theory of network pharmacology indicates that an effect on phenotype may derive from hitting multiple targets, then that combined phenotypic perturbation may be efficacious or deleterious. The major challenge to both drug combination strategies is the regulatory requirement for each individual drug to be shown to be safe as an individual agent and in combination (Hopkins, A. L.; Nat. Chem. Biol.; 2008; 4; 682-690).

An alternative strategy for multitarget therapy is to design a single compound with selective polypharmacology (multi-targeting drug). It has been shown that many approved drugs act on multiple targets. Dosing with a single compound may have advantages over a drug combination in terms of equitable pharmacokinetics and biodistribution. Indeed, troughs in drug exposure due to incompatible pharmacokinetics between components of a combination therapy may create a low-dose window of opportunity where a reduced selection pressure can lead to drug resistance. In terms of drug registration, approval of a single compound acting on multiple targets faces significantly lower regulatory barriers than approval of a combination of new drugs (Hopkins, 2008).

Thus, in a preferred embodiment, the compounds of the present invention having affinity for the α2δ subunits of voltage-gated calcium channels, preferably towards the α2δ-1 subunit of voltage-gated calcium channels, additionally have inhibitory effect towards the μ-receptor and are, thus, more effective to treat chronic pain.

In this way, the present invention relates to compounds having a complementary dual mechanism of action (μ-receptor agonist and blocker of the α₂δ subunit, in particular the α2δ-1 subunit, of voltage-gated calcium channels) which implies a better profile of tolerability than the strong opioids (morphine, oxycodone, fentanyl etc) and/or better efficacy and tolerability than gabapentinoids (pregabalin and gabapentin).

The authors of the present invention have found a serie of compounds that show pharmacological activity towards the α₂δ subunit, in particular the α₂δ-1 subunit, of the voltage-gated calcium channel. The authors of the present invention have also found a serie of compounds that show dual pharmacological activity towards both the α₂δ subunit, in particular the α₂δ-1 subunit, of the voltage-gated calcium channel, and the μ-opioid receptor (MOR or mu-opioid receptor). Both findings resulting in an innovative, effective and alternative solution for the treatment of pain.

In view of the existing results of the currently available therapies and clinical practices, the present invention offers a solution by providing the compounds according to the invention that bind to the α₂δ subunit, in particular the α₂δ-1 subunit, of the voltage-gated calcium channel. Additionally, the present invention offers a solution by combining in a single compound binding to two different targets relevant for the treatment of pain. This was mainly achieved by providing the compounds according to the invention that bind both to the μ-opioid receptor and to the α₂δ subunit, in particular the α₂δ-1 subunit, of the voltage-gated calcium channel.

### SUMMARY OF THE INVENTION

The present invention discloses novel compounds with great affinity to the α2δ subunit of voltage-gated calcium channels, more specifically to the α2δ-1 subunit, and which in preferred embodiments also have inhibitory effect towards the μ-opioid receptor (MOR or mu-oploid receptor), thus resulting in a dual activity for treating pain and pain related disorders.

The main aspect of the present Invention is related to compounds of general formula (i):

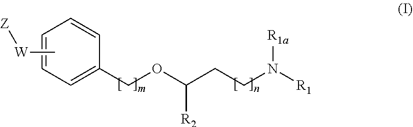

wherein:

R₁ and R₁ₐ are independently from one another a hydrogen atom or a branched or unbranched C₁₋₆ alkyl radical:

R₂ is selected from an optionally substituted 6-membered aryl group and an optionally substituted 5 to 9-membered heteroaryl group having at least one heteroatom selected from the group of N, O and S;

n and m are independently 0, 1 or 2;

—W—Z moiety is in meta or pare position;

W represents —(CH₂)p-, —C(O)— or a bond:

p is 1 or 2;

Z is selected from an optionally substituted 5 to 9-membered heteroaryl group having at least one heteroatom selected from the group of N, O and S; an optionally substituted 3 to 6-membered heterocycloalkyl group having at least one heteroatom selected from the group of N, O and S; and an optionally substituted 5 to 10-membered heterocyclic system having at least one heteroatom selected from the group of N, O and S;

or a pharmaceutically acceptable salt, Isomer, prodrug or solvate thereof.

It is also an aspect of the invention different processes for the preparation of compounds of general formula (I).

Another aspect of the invention refers to the use of such compounds of general formula (I) for the treatment and/or prophylaxis of the α2δ-1 subunit mediated disorders and more preferably for the treatment and/or prophylaxis of disorders mediated by the α2δ-1 subunit of voltage-gated calcium channels and/or the μ-oploid receptor (MOR or mu-opioid receptor). The compounds of the present Invention are particularly suited for the treatment of pain, especially neuropathic pain, and pain related or pain derived conditions.

A further aspect of the invention refers to pharmaceutical compositions comprising one or more compounds of general formula (I) with at least one pharmaceutically acceptable excipient. The pharmaceutical compositions in accordance with the invention can be adapted in order to be administered by any route of administration, be it orally or parenterally, such as pulmonarily, nasally, rectally and/or intravenously, Therefore, the compositions in accordance with the invention may be adapted for topical or systemic application, particularly for dermal, subcutaneous, intramuscular, intra-articular, intraperitoneal, pulmonary, buccal, sublingual, nasal, percutaneous, vaginal, oral or parenteral application.

### DETAILED DESCRIPTION OF THE INVENTION

The invention first relates to compounds of general formula (I)

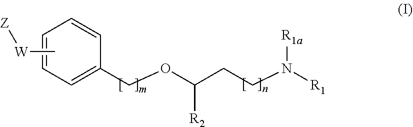

wherein:

R₁ and R₁ₐ are independently from one another a hydrogen atom or a branched or unbranched C₁₋₆ alkyl radical;

R₂ is selected from a 6-membered aryl group optionally substituted by at least one substituent selected from a halogen atom, a branched or unbranched C₁₋₆-alkyl radical, a branched or unbranched C₁₋₆-alkoxy radical, a C₁₋₆-haloalcoxy radical, a C₁₋₆-haloalkyl and a hydroxyl radical; and an optionally substituted 5 to 9-membered heteroaryl group having at least one heteroatom selected from the group of N, O and S optionally substituted by at least one substituent selected from a halogen atom, a branched or unbranched C₁₋₆-alkyl radical, a branched or unbranched C₁₋₆-alkoxy radical, a C₁₋₆-haloalcoxy radical, a C₁₋₆-haloalkyl radical and a hydroxyl radical;

n and m are independently 0, 1 or 2;

—W—Z moiety is in meta or para position;

W represents —(CH₂)p-, —C(O)— or a bond;

p is 1 or 2;

Z is selected from an optionally substituted 5 to 9-membered heteroaryl group having at least one heteroatom selected from the group of N, O and S; an optionally substituted 3 to 6-membered heterocycloalkyl group having at least one heteroatom selected from the group of N, O and S; and an optionally substituted 5 to 10-membered heterocyclic system having at least one heteroatom selected from the group of N, O and S;

or a pharmaceutically acceptable salt, Isomer, prodrug or solvate thereof.

When Z represents a 5 to 9-membered heteroaryl group; a 3 to 6-membered heterocycloalkyl group or a 5 to 10-membered heterocyclic system each of them having at least one heteroatom selected from the group of N, O and S they may be substituted by a ═O group; a branched or unbranched Ca alkyl radical; a phenyl radical in turn optionally substituted by at least one substituent selected from a halogen atom, a branched or unbranched C₁₋₆-alkyl radical, a branched or unbranched C₁₋₆-alkoxy radical, a C₁₋₆-haloalcoxy radical, a C₁₋₆-haloalkyl radical and a hydroxyl radical; a —NR₇ₐR_{7b} radical or a radical:

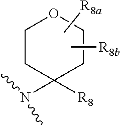

wherein

R₇ₐ, R_{7b} are independently from one another a branched or unbranched C₁₋₆ alkyl radical, a phenyl radical or a —C(O)—C₁₋₆ alkyl radical;

R₈ is selected from a branched or unbranched C₁₋₆ alkyl radical and a —C(O)R₉ radical;

R₈ₐ, R_{8b} are independently from one another a hydrogen atom or a branched or unbranched C₁₋₆ alkyl radical;

R₉ is a 6-membered aryl radical preferably a phenyl, or 5 or 6-membered heteroaryl radical having at least one heteroatom selected from the group of N, O and S, preferably nitrogen, and more preferably a pyridine;

Unless otherwise stated, the compounds of the invention are also meant to include isotopically-labelled forms i.e. compounds which differ only in the presence of one or more isotopically-enriched atoms. For example, compounds having the present structures except for the replacement of at least one hydrogen atom by a deuterium or tritium, or the replacement of at least one carbon by ¹³C- or ¹⁴C-enriched carbon, or the replacement of at least one nitrogen by ¹⁵N-enriched nitrogen are within the scope of this invention.

The compounds of general formula (I) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrugs.

“Halogen” or “halo” as referred in the present Invention represent fluorine, chlorine, bromine or iodine. When the term “halo” is combined with other substituents, such as for instance “C₁₋₆ haloalkyl” or “C₁₋₆ haloalkoxy” it means that the alkyl or alkoxy radical can respectively contain at least one halogen atom.

A leaving group (LG) is a group that in a heteroytic bond cleavage keeps the electron pair of the bond. Suitable leaving groups are well known in the art and include Cl, Br, I and —O—SO₂R′, wherein R′ is F, C₁₋₄-alkyl, C₁₋₄-haloalkyl, or optionally substituted phenyl. The preferred leaving groups are Cl, Br, I, tosylate, mesylate, nosylate, triflate, nonaflate and fluorosulphonate.

“C₁₋₆ alkyl”, as referred to in the present invention, are saturated aliphatic radicals. They may be linear (unbranched) or branched and are optionally substituted. C₁₋₆ alkyl as expressed in the present invention means an alkyl radical of 1, 2, 3, 4, 5 or 6 carbon atoms. Preferred alkyl radicals according to the present invention include but are not restricted to methyl, ethyl, propyl, n-propyl, isopropyl, butyl, n-butyl, tert-butyl, isobutyl, sec-butyl, 1-methylpropyl, 2-methylpropyl, 1,1-dimethylethyl, pentyl, n-pentyl, 1,1-dimethylpropyl, 1,2-dimethylpropyl, 2,2-dimethylpropyl, hexyl or 1-methylpentyl. The most preferred alkyl radical are C₁₋₄ alkyl, such as methyl, ethyl, propyl, n-propyl, isopropyl, butyl, n-butyl, tert-butyl, isobutyl, sec-butyl, 1-methylpropyl, 2-methylpropyl or 1,1-dimethylethyl. Alkyl radicals, as defined in the present invention, are optionally mono- or polysubstituted by substitutents independently selected from a halogen atom, a branched or unbranched C₁₋₆-alkoxy radical, a branched or unbranched C₁₋₆-alkyl radical, a C₁₋₆-haloalcoxy radical, a C₁₋₆-haloalkyl radical, a —CN radical, a trihaloalkyl radical or a hydroxyl radical.

“C₁₋₆ alkoxy” as referred to in the present invention, is understood as meaning an alkyl radical as defined above attached via oxygen linkage to the rest of the molecule. Examples of alkoxy include, but are not limited to methoxy, ethoxy, propoxy, butoxy or tert-butoxy.

“C₃₋₆ Cycloalkyl” as referred to in the present invention, Is understood as meaning saturated and unsaturated (but not aromatic), cyclic hydrocarbons having from 3 to 6 carbon atoms which can optionally be unsubstituted, mono- or polysubstituted. Examples for cycloalkyl radical preferably include but are not restricted to cyclopropyl, cyclobutyl, cyclopentyl or cyclohexyl. Cycloalkyl radicals, as defined in the present invention, are optionally mono- or polysubstituted by substitutents independently selected from a halogen atom, a branched or unbranched C₁₋₆-alkyl radical, a branched or unbranched C₁₋₆-alkoxy radical, a C₁₋₆-haloalcoxy radical, a C₁₋₆-haloalkyl radical, a trihaloalkyl radical and a hydroxyl radical.

“Hetercycloalkyl” as referred to in the present invention, are understood as meaning saturated and unsaturated (but not aromatic), 3 to 6-membered, generally 5 or 6 membered cyclic hydrocarbons which can optionally be unsubstituted, mono- or polysubstituted and which have at least one heteroatom in their structure selected from N, O and S. Examples for heterocycloalkyl radical preferably include but are not restricted to pyrroline, pyrrolidine, pyrazoline, aziridine, azetidine, tetrahydropyrrole, oxirane, oxetane, dioxetane, tetrahydropyrane, tetrahydrofurane, dioxane, dioxolane, oxazolidine, piperidine, piperazine, morpholine, azepane or diazepane. Heterocycloalkyl radicals, as defined in the present invention, are optionally mono- or polysubstituted by substitutents independently selected from a halogen atom, a branched or unbranched C₁₋₆-alkyl radical, a branched or unbranched C₁₋₆-alkoxy radical, a C₁₋₆-haloalkoxy radical, a C₁₋₆-haloalkyl radical, a trihaloalkyl radical and a hydroxyl radical. More preferably heterocycloalkyl in the context of the present invention are 5 or 6-membered ring optionally at least monosubstituted.

“Aryl” as referred to in the present invention, is understood as meaning a ring or ring systems with at least one aromatic ring but without heteroatoms even in only one of the rings. These aryl radicals may optionally be mono- or polysubstituted by substitutents independently selected from a halogen atom, a branched or unbranched C₁₋₆-alkyl, a branched or unbranched C₁₋₆-alkoxy radical, a C₁₋₆-haloalcoxy radical, a C₁₋₆-haloalkyl radical and a hydroxyl radical. Preferred examples of aryl radicals include but are not restricted to phenyl, naphthyl, fluoranthenyl, fluorenyl, tetralinyl, indanyl or anthracenyl radicals, which may optionally be mono- or polysubstituted, if not defined otherwise. More preferably aryl in the context of the present Invention is 6-membered rings optionally at least monosubstituted.

“Heteroaryl” as referred to in the present invention, is understood as a heterocyclic aromatic ring that contains one or more heteroatoms selected from the group consisting of N, O and S and may optionally be mono- or polysubstituted by substituents independently selected from a halogen atom, a branched or unbranched C₁₋₆-alkyl radical, a branched or unbranched C₁₋₆-alkoxy radical, a C₁₋₆-haloalkoxy radical, a C₁₋₆-haloalkyl radical, a trihaloalkyl radical and a hydroxyl radical. Preferred examples of heteroaryls include but are not restricted to furan, thiophene, thiazole, pyrrole, pyridine, pyrimidine, pyridazine, pyrazine, triazole, pyrazole, imidazole, isoxazole or oxadiazole. More preferably heteroaryl in the context of the present invention are 5 or 6-membered rings optionally at least monosubstituted.

“Heterocyclic system”, as defined in the present invention, comprise any saturated, unsaturated or aromatic carbocyclic ring systems which are optionally at least mono-substituted and which contain at least one heteroatom as ring member. Preferred heteroatoms for these heterocyclyl radicals are N, S or O. Preferred substituents for heterocyclyl radicals, according to the present invention, are F, Cl, Br, I, NH₂, SH, OH, SO₂, CF₃, carboxy, amido, cyano, carbamyl, nitro, phenyl, benzyl, —SONH₂, branched or unbranched C₁₋₆ alkyl and/or branched or unbranched C₁₋₆-alkoxy. Preferred examples of heteroaryls include but are not restricted to benzofuran, quinoline, isoquinoline, phthalazine, indole, benzotriazole, benzodioxolane, benzodioxane, benzimidazole, carbazole or quinazoline.

The term “C₁₋₃ alkylene” is understood as meaning a divalent alkyl group like —CH₂— or —CH₂—CH₂— or —CH₂—CH₂—CH₂—.

The term “ring system” according to the present invention refers to a system consisting of at least two or more rings of connected atoms which are joined with “joined” meaning that the respective rings are sharing one (like a spiro structure), two or more atoms being a member or members of both joined rings. The “ring system” thus defined comprises saturated, unsaturated or aromatic carbocyclic rings which contain optionally at least one heteroatom as ring member and which are optionally at least mono-substituted and may be joined to other carbocyclic ring systems such as aryl radicals, heteroaryl radicals, cycloalkyl radicals etc.

The terms “condensed”, “annulated” or “annelated” are also used by those skilled in the art to designate this kind of join.

The term “salt” is to be understood as meaning any form of the active compound according to the invention in which this assumes an ionic form or is charged and is coupled with a counter-ion (a cation or anion) or is in solution. By this are also to be understood complexes of the active compound with other molecules and ions, in particular complexes which are complexed via ionic interactions. The definition particularly includes physiologically acceptable salts, this term must be understood as equivalent to “pharmacologically acceptable salts”.

The term “pharmaceutically acceptable salts” in the context of this invention means any salt that is tolerated physiologically (normally meaning that it is not toxic, particularly as a result of the counter-ion) when used in an appropriate manner for a treatment, particularly applied or used in humans and/or mammals. These physiologically acceptable salts may be formed with cations or bases and, in the context of this invention, are understood to be salts formed by at least one compound used in accordance with the invention—normally an acid (deprotonated)—such as an anion and at least one physiologically tolerated cation, preferably Inorganic, particularly when used on humans and/or mammals. Salts with alkali and alkali earth metals are particularly preferred, as well as those formed with ammonium cations (NH₄⁺). Preferred salts are those formed with (mono) or (di)sodium, (mono) or (dl)potassium, magnesium or calcium. These physiologically acceptable salts may also be formed with anions or acids and, in the context of this invention, are understood as being salts formed by at least one compound used in accordance with the invention—normally protonated, for example in nitrogen—such as a cation and at least one physiologically tolerated anion, particularly when used on humans and/or mammals. This definition specifically includes in the context of this invention a salt formed by a physiologically tolerated acid, i.e. salts of a specific active compound with physiologically tolerated organic or Inorganic acids—particularly when used on humans and/or mammals. Examples of this type of salts are those formed with: hydrochloric acid, hydrobromic acid, sulphuric acid, methanesulfonic acid, formic acid, acetic acid, oxalic acid, succinic acid, malic acid, tartaric acid, mandelic acid, fumaric acid, lactic acid or citric acid.

The term “solvate” is to be understood as meaning any form of the active compound according to the invention in which this compound has attached to it via non-covalent binding another molecule (most likely a polar solvent) especially including hydrates and alcoholates, e.g. methanolate.

The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the compounds of the invention: esters, amino acid esters, phosphate esters, metal salts sulfonate esters, carbamates, and amides. Examples of well known methods of producing a prodrug of a given acting compound are known to those skilled in the art and can be found e.g. in Krogsgaard-Larsen et al. “Textbook of Drug design and Discovery” Taylor & Francis (April 2002).

Any compound that is a prodrug of a compound of general formula (I) is within the scope of the invention. Particularly favored prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

In a particular and preferred embodiment of the invention, R₁ₐ represents a hydrogen atom.

In another particular and preferred embodiment of the invention, R₁ represents a branched or unbranched C₁₋₆alkyl radical, more preferable methyl.

In a particularly preferred embodiment of the invention, R₁ₐ represents a hydrogen atom and R₁ represents a methyl.

In another particular and preferred embodiment of the invention, R₂ represents a thiophene or a phenyl. These groups may optionally substituted by at least one substituent selected from a halogen atom, a branched or unbranched C₁₋₆ alkyl radical, a branched or unbranched C₁₋₆-alkoxy radical, a C₁₋₆-haloalcoxy radical, a C₁₋₆-haloalkyl radical and a hydroxyl radical. The thiophene radical can be attached to the main structure through different point of attachment. For instance, it might be a 2-thiophene or 3-thiophene.

In a particularly preferred embodiment R₂ represents a group selected from:

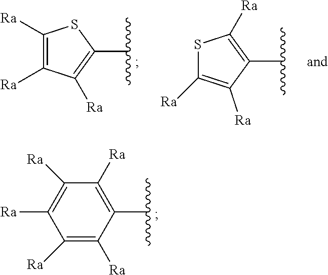

wherein each Rₐ independently represents a hydrogen atom, a halogen atom, a branched or unbranched C₁₋₆ alkyl radical, a branched or unbranched C₁₋₆-alkoxy radical, a C₁₋₆-haloalcoxy radical, a C₁₋₆-haloalkyl radical or a hydroxyl radical.

In a still more particularly preferred embodiment R₂ represents a group selected from:

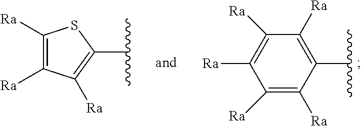

wherein each Rₐ independently represents a hydrogen atom, a halogen atom, a branched or unbranched C₁₋₆ alkyl radical, a branched or unbranched C₁₋₆-alkoxy radical, a C₁₋₆-haloalcoxy radical, a C₁₋₆-haloalkyl radical or a hydroxyl radical.

In another particular and preferred embodiment, Z is selected from:

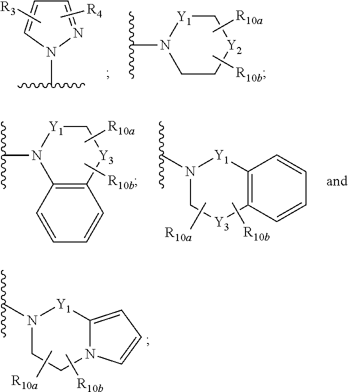

wherein:

R₃ is —O—, —NR₅— or —CR₆R₇—,

R₅ is a hydrogen atom or a branched or unbranched C₁₋₆alkyl radical;

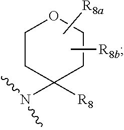

R₆, R₇ are independently from one another a hydrogen atom, a phenyl radical optionally substituted or a NR₇ₐR_{7b} radical;

R₇ₐ, R_{7b} are independently from one another a branched or unbranched C₁₋₆ alkyl radical, a phenyl radical optionally substituted or a —C(O)—C₁₋₆ alkyl radical;

R₈ is selected from a branched or unbranched C₁₋₆ alkyl radical and a C(O)R₉ radical;

R₈ₐ, R_{8b} are independently from one another a hydrogen atom or a branched or unbranched C₁₋₆ alkyl radical;

R₉ is an optionally substituted 6-membered aryl radical or an optionally substituted 5 or 6-membered heteroaryl group having at least one heteroatom selected from the group of N, O and S, preferably Nitrogen;

R₁₀ₐ, R_{10b} are independently from one another a hydrogen atom or a branched or unbranched C₁₋₆ alkyl radical;

or alternatively R₁₀ₐ or R_{10b} forms together with Y₁, Y₂, a carbon atom and the carbon atoms to which they are attached a substituted or unsubstituted aryl radical or heteroaryl radical having at least one heteroatom selected from the group of N, O and S.

In a still more particularly preferred embodiment Z represents a group selected from:

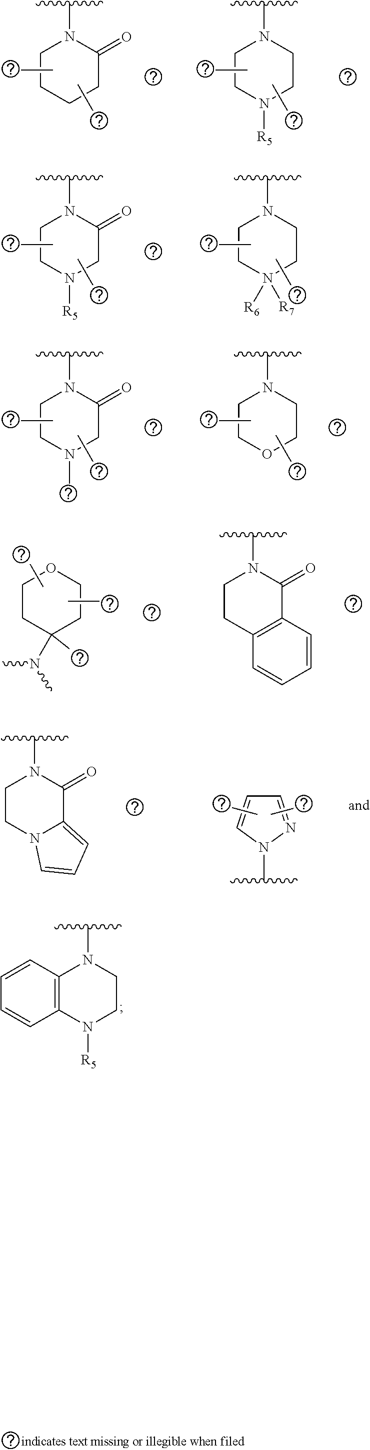

wherein R₃, R₄, R₅, R₇, R₈, R₈ₐ, R_{8b}, R₁₀ₐ and R_{10b} are as defined above.

A particular and preferred embodiment of the invention is represented by a compound of general formula (I):

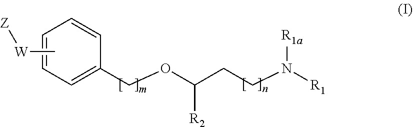

wherein

R₁ₐ represents a hydrogen atom;

R₁ represents a branched or unbranched C₁₋₆ alkyl radical, more preferable methyl;

R₂ represents a radical selected from:

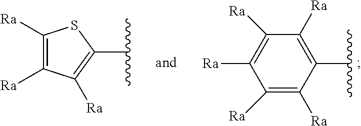

wherein each R₆ independently represents a hydrogen atom, a halogen atom, a branched or unbranched C₁₋₆ alkyl radical, a branched or unbranched Ca alkoxy radical, a C₁₋₆-haloalcoxy radical, a C₁₋₆-haloalkyl radical or a hydroxyl radical;

Z is selected from:

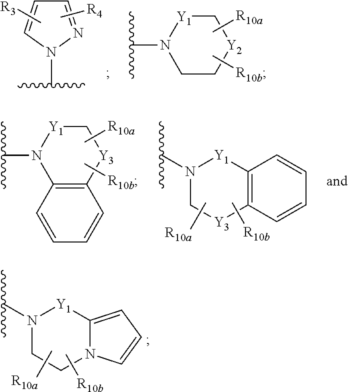

wherein Y₁, Y₂, Y₃, R₃, R₄, R₁₀ₐ and R_{10b} have the above defined meaning; or a pharmaceutically acceptable salt, isomer, prodrug or solvate thereof.

Another particular and preferred embodiment of the invention is represented by a compound of general formula (I):

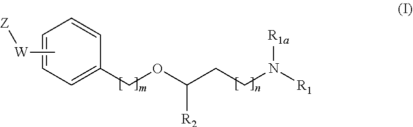

wherein

R₁ₐ represents a hydrogen atom;

R₁ represents a branched or unbranched C₁₋₆ alkyl radical, more preferable methyl;

R₂ represents a radical selected from:

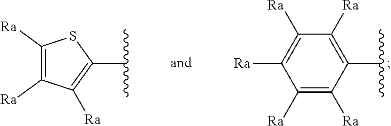

wherein each Rₐ independently represents a hydrogen atom, a halogen atom, a branched or unbranched C₁₋₆ alkyl radical, a branched or unbranched C₁₋₆ alkoxy radical, a C₁₋₆-haloalkoxy radical, a C₁₋₆-haloalkyl radical or a hydroxyl radical;

Z is selected from:

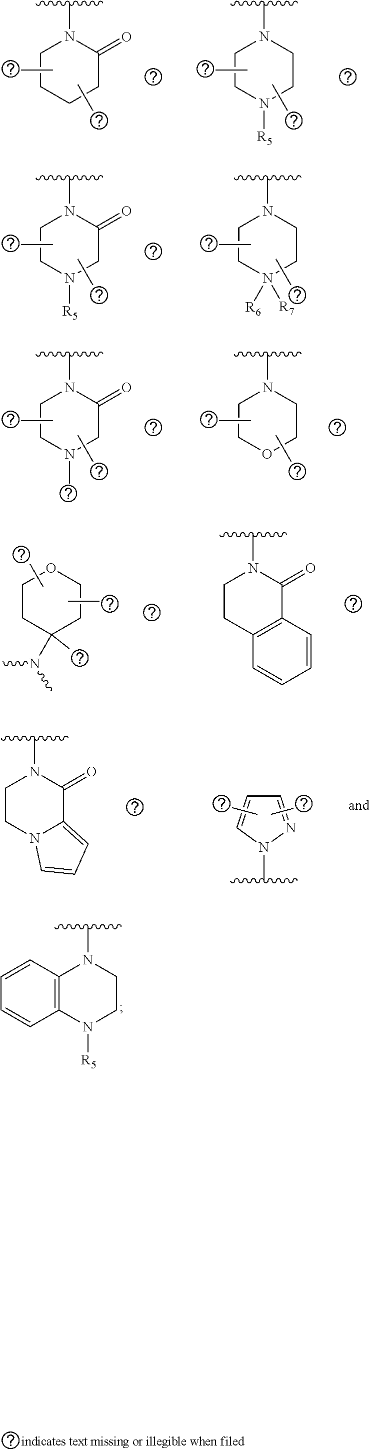

wherein R₃, R₄, R₅, R₆, R₇, R₈, R₈ₐ, R_{8b}, R₁₀ₐ and R_{10b} are as defined above;

or a pharmaceutically acceptable salt, isomer, prodrug or solvate thereof,

A further embodiment of the invention is related to compounds of general formula (I) having the following subformula (Iaa) or (Iab):

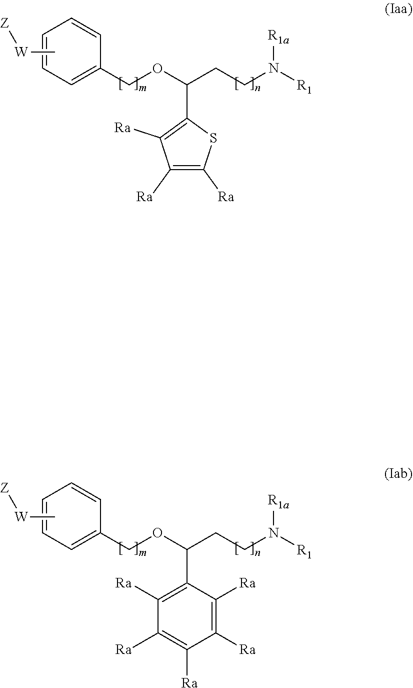

wherein Rₐ, R₁, R₁ₐ, m, n, W and Z are as defined above;

or a pharmaceutically acceptable salt, isomer, prodrug or solvate thereof.

Still another embodiment of the invention is related to compounds of general formula (I) having the following subformula (Iba), (Ibb) or (Ibc):

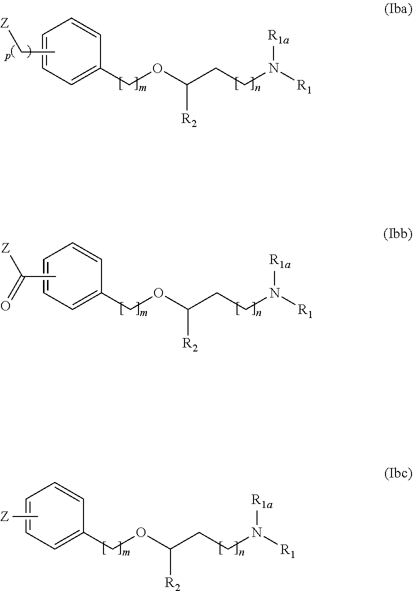

wherein R₁, R₁ₐ, R₂, m, n, p, and Z are as defined above;

or a pharmaceutically acceptable salt, isomer, prodrug or solvate thereof.

Still another embodiment of the invention is related to compounds of general formula (I) having the following subformula (Ica) or (Icb);

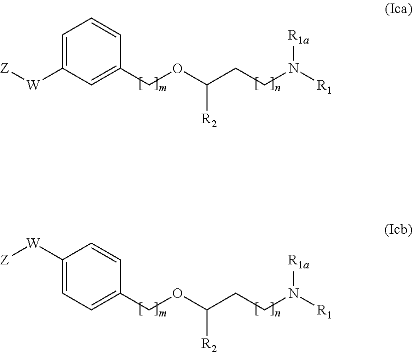

wherein R₁, R₁ₐ, R₂, m, n, W and Z are as defined above;

or a pharmaceutically acceptable salt, isomer, prodrug or solvate thereof.

The compounds of the present invention represented by the above described formula (i) may include enantiomers depending on the presence of chiral centers or isomers depending on the presence of double bonds (e.g. Z, E). The single isomers, enantiomers or diastereoisomers and mixtures thereof fall within the scope of the present invention.

Among all the compounds described in the general formula (I), the following compounds are preferred for showing and intense inhibitory effect towards the subunit 25-1 of voltage-gated calcium channels (VGCC):
[1] 3-(3-(3-((3,5-Dimethylpiperazin-1-yl methyl)phenoxy)-N-methyl-3-(thiophen-2-yl)propan-1-amine;
[2] N,N-dimethyl-1-(3-(3-(methylamino)-1-(thiophen-2-yl)propoxy)benzyl)-4-phenylpiperidin-4-amine;
[3] 3-(1-(3-(3-(Methylamino)-1-(thiophen-2-yl)propoxy)benzyl)piperidin-4-yl)phenol;
[4] N-(1-(3-(3-(methylamino)-1-(thiophen-2-yl)propoxy)benzyl)piperidin-4-yl)-N-phenylpropionamide;
[5] 3-(4-(Dimethylamino)-1-(3-(3-(methylamino)-1-(thiophen-2-yl)propoxy)benzyl)piperidin-4-yl)phenol;
[6] N-methyl-3-(3-(piperazin-1-ylmethyl)phenoxy)-3-(thiophen-2-yl)propan-1-amine;
[7] (4-(Dimethylamino)-4-phenylpiperidin-1-yl)(3-(3-(methylamino)-1-(thiophen-2-yl)propoxy)phenyl)methanone;
[8] (3-(3-(Methylamino)-1-phenylpropoxy)phenyl)(piperidin-1-yl)methanone;
[9] (3-(3-(Methylamino)-1-phenylpropoxy)phenyl)(4-methylpiperazin-1-yl)methanone;
[10] (3-(3-(Methylamino)-1-phenylpropoxy)phenyl)(morpholino)methanone;
[11] (3-(3-(Methylamino)-1-(thiophen-2-yl)propoxy)phenyl)(4-methylpiperazin-1-yl)methanone;
[12a] (R)-3-(3-(2-(4 ((S)-4-ethyl-2,2-dimethyltetrahydro-2H-pyran-4-yl)piperazin-1-yl)ethyl)phenoxy)-N-methyl-3-phenylpropan-1-amine;
[12b] (R)-3-(3-(2-(4-((R)-4-ethyl-2,2-dimethyltetrahydro-2H-pyran-4-yl)piperazin-1-yl)ethyl)phenoxy)-N-methyl-3-phenylpropan-1-amine;
[13] 3-(3-(3,5-Dimethyl-1H-pyrazol-1-yl)phenoxy)-N-methyl-3-phenyl propan-1-amine;
[14] (S)-3-(4-(dimethylamino)-1-(3-(3-(methylamino)-1-(thiophen-2-yl) propoxy)benzyl)piperidin-4-yl)phenol;
[15] (S)-N,N-dimethyl-1-(3-(3-(methylamino)-1-(thiophen-2-yl)propoxy)benzyl)-4-phenylpiperidin-4-amine;
[16] (R)-N,N-dimethyl-1-(3-(3-(methylamino)-1-(thiophen-2-yl)propoxy)benzyl)-4-phenylpiperidin-4-amine;
[17] (R)-3-(4-(dimethylamino)-1-(3-(3-(methylamino)-1-(thiophen-2-yl)propoxy)benzyl)piperidin-4-yl)phenol;
[18] ((R)-2,2-dimethyl-4-(4-(3-((S)-3-(methylamino)-1-(thiophen-2-yl)propoxy)benzyl)piperazin-1-yl)tetrahydro-2H-pyran-4-yl)(pyridin-2-yl)methanone;
[19] ((S)-2,2-dimethyl-4-(4-(3-((S)-3-(methylamino)-1-(thiophen-2-yl)propoxy)benzyl)piperazin-1-yl)tetrahydro-2H-pyran-4-yl)(pyridin-2-yl)methanone;
[20] N,N-dimethyl-1-(3-((3-(methylamino)-1-(thiophen-2-yl)propoxymethyl)benzyl)-4-phenylpiperidin-4-amine;
[21] (S)-N,N-dimethyl-1-(3-((3-(methylamino)-1-(thiophen-2-yl)propoxy)methyl)benzyl)-4-phenylpiperidin-4-amine;
[22] (R)-N,N-dimethyl-1-(3-((3-(methylamino)-1-(thiophen-2-yl)propoxy)methyl)benzyl)-4-phenylpiperidin-4-amine;
[23] (S)-3-(4-(dimethylamino)-1-(3-((3-(methylamino)-1-(thiophen-2-yl)propoxy)methyl)benzyl)piperidin-4-yl)phenol;
[24] (R)-3-(4-(dimethylamino)-1-(3-((3-(methylamino)-1-(thiophen-2-yl)propoxy)methyl)benzyl)piperidin-4-yl)phenol;
[25] N,N-dimethyl-1-(3-((3-(methylamino)-1-phenylpropoxy)methyl)benzyl)-4-phenylpiperidin-4-amine;
[26] (R)-N,N-dimethyl-1-(3-((3-(methylamino)-1-phenylpropoxy)methyl)benzyl)-4-phenylpiperidin-4-amine;
[27] (S)-N,N-dimethyl-1-(3-((3-(methylamino)-1-phenylpropoxy)methyl)benzyl)-4-phenylpiperidin-4-amine;
[28] ((S)-2,2-dimethyl-4-(4-(3-(((R)-3-(methylamino)-1-phenylpropoxy)methyl)benzyl)piperazin-1-yl)tetrahydro-2H-pyran-4-yl)(pyridin-2-yl)methanone;
[29] ((R)-2,2-dimethyl-4-(4-(3-(((R)-3-(methylamino)-1-phenylpropoxy)methyl)benzyl)piperazin-1-yl)tetrahydro-2H-pyran-4-yl)(pyridin-2-yl)methanone;
[30] (R)-3-((3-((4-((R)-4-ethyl-2,2-dimethyltetrahydro-2H-pyran-4-yl)piperazin-1-yl)methyl)benzyl)oxy)-N-methyl-3-phenylpropan-1-amine;
[31] (R)-3-((3-((4-((S)-4-ethyl-2,2-dimethyltetrahydro-2H-pyran-4-yl)piperazin-1-yl)methyl)benzyl)oxy)-N-methyl-3-phenylpropan-1-amine;
[32] 2-(3-((3-(Methylamino)-1-(thiophen-2-yl)propoxy)methyl)phenyl)-3,4-dihydroisoquinolin-1(2H)-one;
[33] 1-(3-(3-(Methylamino)-1-phenylpropoxy)phenyl)piperidin-2-one;
[34] 1-(3-(3-(Methylamino)-1-(thiophen-2-yl)propoxy)phenyl)piperidin-2-one;
[35] N-methyl-3-(3-(4-methylpiperazin-1-yl)phenoxy)-3-phenylpropan-1-amine;
[36] 4-Methyl-1-(3-(3-(methylamino)-1-(thiophen-2-yl)propoxy)phenyl)piperazin-2-one;
[37] N-methyl-3-(3-(4-methylpiperazin-1-yl)phenoxy)-3-(thiophen-2-yl)propan-1-amine;
[38] 1-(3-((3-(Methylamino)-1-phenylpropoxy)methyl)phenyl)piperidin-2-one;
[39] 1-(3-((3-(Methylamino)-1-(thiophen-2-yl)propoxy)methyl)phenyl)piperidin-2-one;
[40] N-methyl-3-((3-(4-methylpiperazin-1-yl)benzyl)oxy)-3-phenylpropan-1-amine;
[41] 4-Methyl-1-(3-((3-(methylamino)-1-phenylpropoxy)methyl)phenyl)piperazin-2-one;
[42] N-methyl-3-(3-(piperazin-1-yl)phenoxy)-3-(thiophen-2-yl)propan-1-amine;
[43] N,N-dimethyl-1-(3-(3-(methylamino)-1-(thiophen-2-yl)propoxy)phenyl)-4-phenylpiperidin-4-amine;
[44] 3-(3-((3S,5R)-3,5-dimethylpiperazin-1-yl)phenoxy)-N-methyl-3-(thiophen-2-yl)propan-1-amine;
[45] N,N-dimethyl-1-(3-((3-(methylamino-1-(thiophen-2-yl)propoxy)methyl)phenyl)-4-phenylpiperidin-4-amine;
[46] N,N-dimethyl-1-(3-((3-(methylamino)-1-phenylpropoxy)methyl)phenyl)-4-phenylpiperidin-4-amine;
[47] 3-((3-((3,4-Dihydroquinoxalin-1(2H)-yl)benzyl)oxy)-N-methyl-3-phenylpropan-1-amine;
[48] N,N-dimethyl-1-(3-((3-(methylamino)-1-phenylpropoxy)methyl)phenyl)piperidin-4-amine;
[49] N,N-dimethyl-1-(4-((3-(methylamino)-1-(thiophen-2-yl)propoxy)methyl)phenyl)-4-phenylpiperidin-4-amine;
[50] (S)-2-(4-(3-(methylamino)-1-(thiophen-2-yl)propoxy)benzyl)-3,4-dihydroisoquinolin-1(2H)-one;
[51] N,N-dimethyl-1-(4(3-(3-(methylamino)-1-(thiophen-2-yl)propoxy)benzyl)-4-phenylpiperidin-4-amine;
[52] N-methyl-3-(4-((4-methylpiperazin-1-yl)methyl)phenoxy-3-(thiophen-2-yl)propan-1-amine;
[53] (S)-2-(4-(3-(methylamino)-1-(thiophen-2-yl)propoxy)benzyl)-3,4-dihydropyrrolo[1,2-a]pyrazin-1(2H)-one;
[56] (4-(Dimethylamino)-4-phenylpiperidin-1-yl)(4-(3-(methylamino)-1-(thiophen-2-yl)propoxy)phenyl)methanone;
[57] N,N-dim ethyl-1-(4-(3-(methylamino)-1-(thiophen-2-yl)propoxy)phenethyl)-4-phenylpiperidin-4-amine and
[58] N-(1-(3-(3-(methylamino)-1-(thiophen-2-yl)propoxy)phenethyl)piperidin-4-yl)-N-phenylpropionamide;

or a pharmaceutically acceptable salt, isomer, prodrug or solvate thereof.

Among compounds of general formula (I) some subgroups of compounds have shown in addition a dual affinity towards the subunit α2δ-1 of voltage-gated calcium channels (VGCC) and the μ-opioid receptor (MOR or mu-opioid receptor). These compounds having dual affinity represent the preferred embodiments of the invention and are represented among one of the following of formula (Id), (Ie), (If), (Ig), (Ih), (Ii) or (Ij).

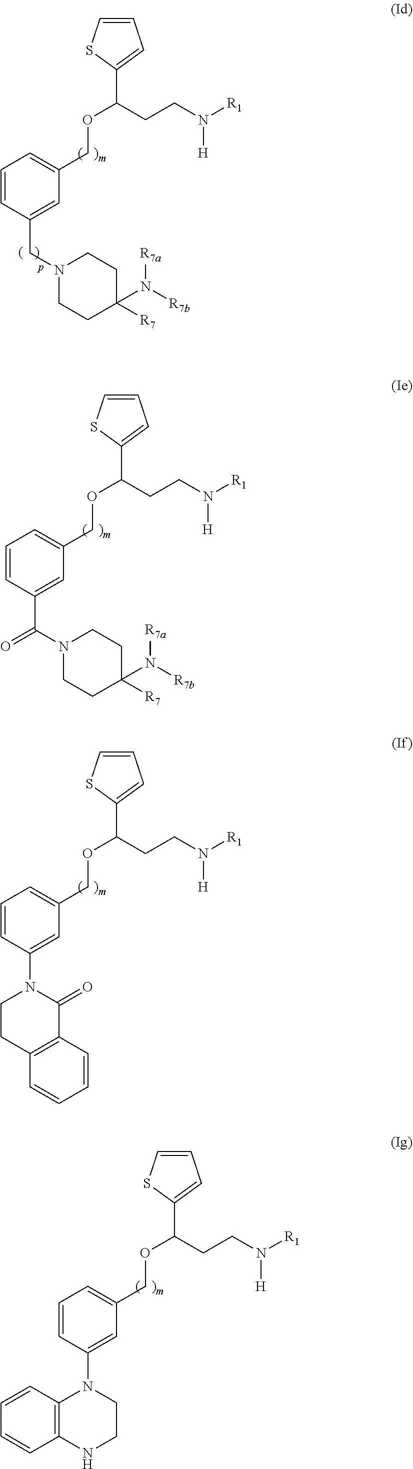

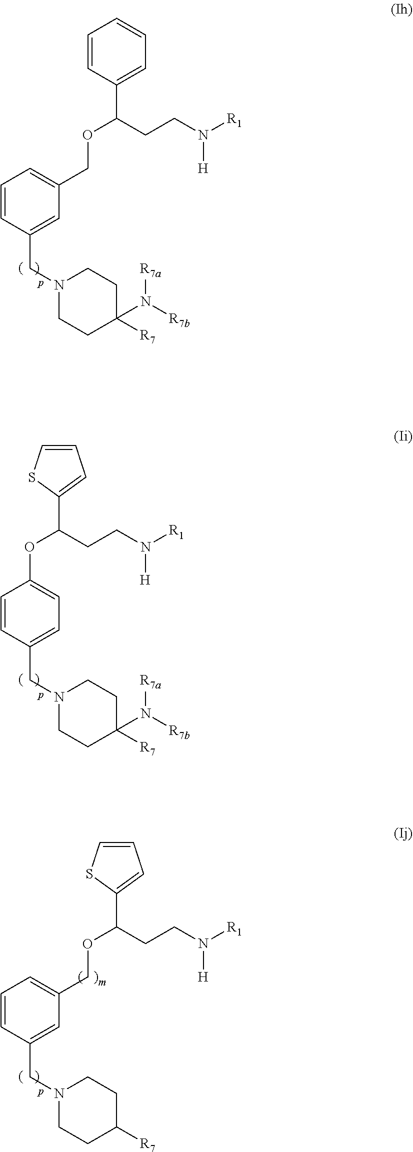

wherein R₁, m, p, R₇, R₇ₐ and R_{7b} are as defined before for general formula (I).

The preferred compounds of the invention showing dual inhibitory effect towards the subunit α2δ-1 of voltage-gated calcium channels (VGCC) and the μ-opioid receptor (MOR or mu-opioid receptor) are selected from the following group:
[2] N,N-dimethyl-1(3-(3-(methylamino)-1-(thiophen-2-yl)propoxy)benzyl)-4-phenylpiperidin-4-amine;
[3] 3-(1-(3-(3-(Methylamino)-1-(thiophen-2-yl)propoxy)benzyl)piperidin-4-yl)phenol;
[4] N-(1-(3-(3-(methylamino)-1-(thiophen-2-yl)propoxy)benzyl)piperidin-4-yl)-N-phenylpropionamide;
[5] 3-(4-(Dimethylamino)-1-(3-(3-(methylamino)-1-(thiophen-2-yl)propoxy)benzyl)piperidin-4-yl)phenol;
[7] (4-(Dimethylamino)-4-phenylpiperidin-1-yl)(3-(3-(methylamino)-1-(thiophen-2-yl)propoxy)phenyl)methanone;
[14] (S)-3-(4-(dimethylamino)-1-(3-(3-(methylamino)-1-(thiophen-2-yl) propoxy)benzyl)piperidin-4-yl)phenol;
[15] (S)-N,N-dimethyl-1-(3-(3-(methylamino)-1-(thiophen-2-yl)propoxy)benzyl)-4-phenylpiperidin-4-amine;
[16] (R)-N,N-dimethyl-1-(3-(3-(methylamino)-1-(thiophen-2-yl)propoxy)benzyl)-4-phenylpiperidin-4-amine;
[17] (R)-3-(4-(dimethylamino)-1-(3-(3-(methylamino)-1-(thiophen-2-yl)propoxy)benzyl)piperidin-4-yl)phenol;
[20] N,N-dimethyl-1-(3-((3-(methylamino)-1-(thiophen-2-yl)propoxy)methyl)benzyl)-4-phenylpiperidin-4-amine;
[21] (S)-N,N-dimethyl-1-(3-((3-(methylamino)-1-(thiophen-2-yl)propoxy)methyl)benzyl)-4-phenylpiperidin-4-amine;
[22] (R)-N,N-dimethyl-1-(3-((3-(methylamino)-1-(thiophen-2-yl)propoxy)methyl)benzyl)-4-phenylpiperidin-4-amine;
[23] (S)-3-(4-(dimethylamino)-1-(3-((3-(methylamino)-1-(thiophen-2-yl)propoxy)methyl)benzyl)piperidin-4-1)phenol;
[24] (R)-3-(4-(dimethylamino)-1-(3-((3-(methylamino)-1-(thiophen-2-yl)propoxy)methyl)benzyl)piperidin-4-yl)phenol;
[25] N,N-dimethyl-1-(3-((3-(methylamino)-1-phenylpropoxy)methy)benzyl)-4-phenylpiperidin-4-amine;
[26] (R)-N,N-dimethyl-1-(3-((3-(methylamino)-1-phenylpropoxy)methy)benzyl)-4-phenylpyridin-4-amine;
[27] (S)-N,N-dimethyl-1-(3-((3-(methylamino)-1-phenylpropoxy)methyl)benzyl)-4-phenylpiperidin-4-amine;
[43] N,N-dimethyl-1-(3-(3-(methylamino)-1-(thiophen-2-yl)propoxy)phenyl)-4-phenylpiperidin-4-amine;
[45] N,N-dimethyl-1-(3-((3-(methylamino)-1-(thiophen-2-yl)propoxy)methyl)phenyl)-4-phenylpiperidin-4-amine;
[46] N,N-dimethyl-1-(3-((3-(methylamino)-1-phenylpropoxy)methy)phenyl)-4-phenylpiperidin-4-amine;
[47] 3-((3-(3,4-Dihydroquinoxalin-1(2H)-yl)benzyl)oxy)-N-methyl-3-phenylpropan-1-amine and
[51] N,N-dimenthyl-1-(4-(3-(methylamino)-1-(thiophen-2-yl)propoxy)benzyl)-4-phenylpiperidin-4-amine;

or a pharmaceutically acceptable salt, Isomer, prodrug or solvate thereof.

In another preferred embodiment, the compounds showing a dual affinity towards the subunit α2δ-1 of voltage-gated calcium channels (VGCC) and the μ-opioid receptor (MOR or mu-opioid receptor) are selected from:
[54] 3-(4-((3,4-Dihydroquinolin-1(2H)-yl)methyl)-3-fluorophenoxy)-N-methyl-3-(thiophen-2-yl)propan 1-amine and
[55] 3-(4-((3,4-Dihydroisoquinolin-2(1H)-yl)methyl)-3-fluorophenoxy)-N-methyl-3-(thiophen-2-yl)propan-1-amine:

or a pharmaceutically acceptable salt, isomer, prodrug or solvate thereof.

In another aspect, the invention refers to the processes for the preparation of the compounds of general formula (I):

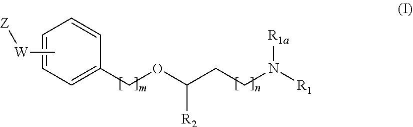

Thus, a process is described for the preparation of compounds of general formula (I) wherein m is 0 (Method A for synthesizing compounds of formula IA) and compounds of general formula (I) wherein m is 1 (Method B for synthesizing compounds of formula IB) starting from a compound of formula II, as shown in the following Scheme 1:

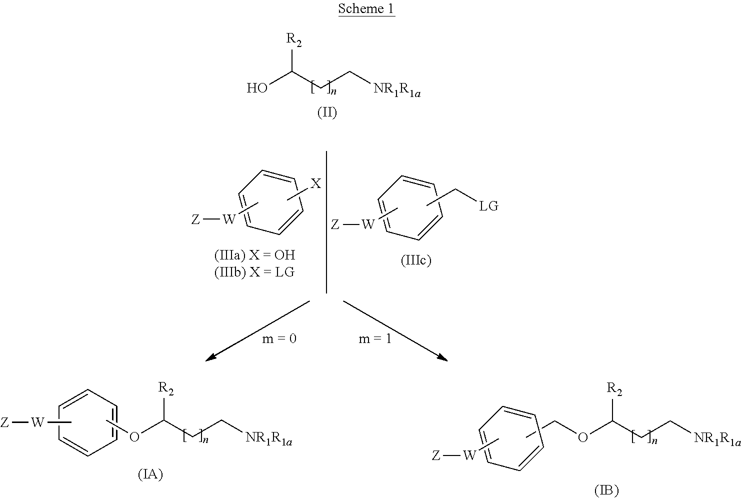

wherein R₁, R₁ₐ, R₂, W, Z and n are as defined before, LG represents a leaving group (such as chloro, bromo, iodo, mesylate, tosylate, nosylate or triflate and X can be OH or LG.

Depending on the meaning of m, different reaction pathways will apply starting from a compound of formula (I). For m=0, the compounds of formula (IA) can be prepared following Method A. For m=1, the compounds of formula (IB) can be prepared following Method B.

### Method A

A compound of formula (IA) can be prepared by reacting a compound of formula (II)

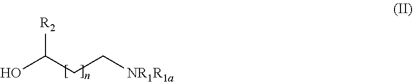

with a suitable compound of formula (IIIa) or (IIIb)

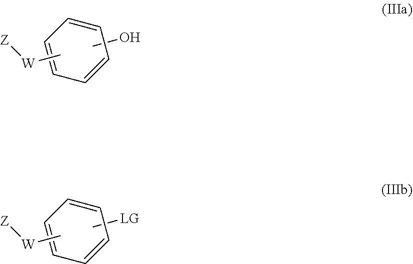

in which case different reaction conditions will apply:
a) When a hydroxy compound of formula (IIIa) is used, the reaction is carried out under conventional Mitsunobu conditions by treating an alcohol of formula (II) with a compound of formula (IIIa) in the presence of an azo compound such as 1,1′-(azodicarbonyl)dipiperidine (ADDP), diisopropylazodicarboxylate (DIAD) or diethyl azodicarboxylate (DEAD) and a phosphine such as tributylphosphine or triphenylphosphine. The Mitsunobu reaction is carried out in a suitable solvent, such as toluene or tetrahydrofuran (THF); at a suitable temperature comprised between 0° C. and the reflux temperature, preferably at room temperature, or alternatively, the reactions can be carried out in a microwave reactor.
b) When a compound of formula (IIIb) is used, the reaction is carried out under conventional aromatic nucleophilic substitution conditions by treating an alcohol of formula (II) with a compound of formula (IIIb) wherein LG represents a leaving group (preferably fluoro), in the presence of a strong base such as sodium hydride. The reaction is carried out in a suitable solvent, such as a polar aprotic solvent, preferably dimethylformamide (DMF) or dimethylacetamide; at a suitable temperature comprised between room temperature and the reflux temperature, preferably heating, or alternatively, the reactions can be carried out in a microwave reactor. Alternatively, when LG is triflate, bromo or iodo, the compound of formula (IIIb) can be introduced under cross-coupling conditions, using a Pd or Cu catalyst and a suitable ligand.

### Method B

A compound of formula (IB) can be prepared by reacting a compound of formula (II)

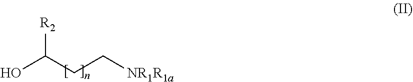

with an alkylating agent of formula (IIIc)

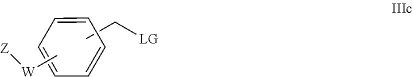

The reaction is preferably carried out in the presence of a strong base such as sodium hydride or potassium tert-butoxide. The alkylation reaction is carried out in a suitable solvent, such as tetrahydrofuran or dimethylformamide, at a suitable temperature comprised between room temperature and the reflux temperature, preferably heating, or alternatively, the reactions can be carried out in a microwave reactor. Additionally, an activating agent such as sodium iodide or a phase transfer catalyst such as tetrabutylammonium iodide can be used.

Alternatively, either in method A or B, the amino group NR₁R₁ₑ can be incorporated at any step of the synthesis by reaction of a compound of formula (II-LG), (IV-LG) or (V-LG) wherein LG represents a leaving group (such as chloro, bromo, iodo, mesylate, tosylate, nosylate or triflate) with an amine of formula (VII), as shown in Scheme 2 below. The alkylation reaction is carried out in a suitable solvent, such as ethanol, dimethylformamide, dimethylsulfoxide (DMSO), acetonitrile (ACN) or a mixture of an organic solvent and water, preferably ethanol; optionally in the presence of a base such as K₂CO₃ or triethylamine (TEA); at a suitable temperature comprised between room temperature and the reflux temperature, preferably heating, or alternatively, the reactions can be carried out in a microwave reactor. Additionally, an activating agent such as sodium iodide or potassium iodide can be used.

Additionally, it may be necessary to protect the amino group —NR₁R₁ₑ or other reactive or labile groups present in the molecules with any suitable protecting group (P), as also shown in scheme 2, such as for example Boc (tert-butoxycarbonyl) or Teoc (2-(trimethylsilyl)ethoxycarbonyl). The procedures for the introduction and removal of these protecting groups are well known in the art and can be found thoroughly described in the literature. For example using di-tert-butyl dicarbonate or 4-nitrophenyl (2-(trimethylsilyl)ethyl)carbonate, in an organic solvent, preferably dichloromethane (DCM), at a temperature range of 0-60° C. Alternatively, in the presence of a base, preferably N,N-disopropylethylamine (DIPEA) or TEA. Boc or Teoc deprotection can be effected by any suitable method, such as treatment with an acid, preferably HCl or trifluoroacetic acid in an appropriate solvent such as 1,4-dioxane. DCM, ethyl acetate or a mixture of an organic solvent and water alternatively by treatment with ZnBr₂ in an organic solvent, preferably DCM; alternatively, for Teoc deprotection, by reaction with CsF in an organic solvent, preferably DMF at a temperature range of 20-130° C., alternatively under microwaves irradiation.

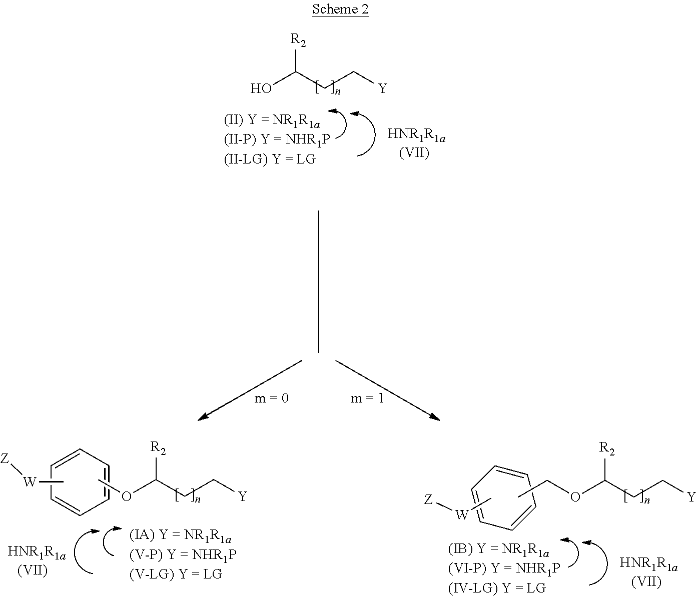

### Method C

Alternatively, the compounds of general formula (I)

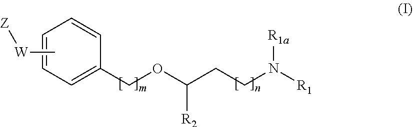

(or its counterparts V/VI-P and V/VI-LG, respectively) can be prepared starting from an intermediate compound (VIII) (or its counterparts VIII-P and VIII-LG)

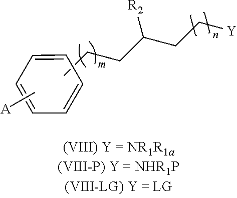

by introducing the substituents Z—W— at any step during the synthesis, from any suitable group A as described in Scheme 3.

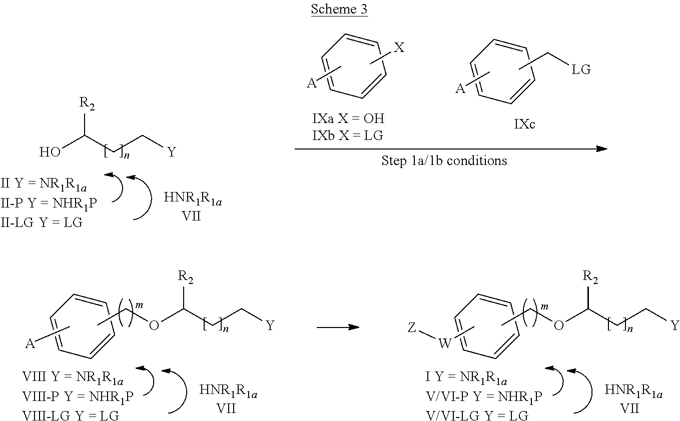

wherein R₁, R₁ₐ, R₂, W, Z, m and n have the meanings as defined above, LG represents a leaving group (such as chloro, bromo, iodo, mesylate, tosylate, nosylate or triflate). P represents a protecting group of the amino function and A represents a suitable function to be converted to a group Z—W—. In particular A may represent an aldehyde, a carboxylic acid, or a suitable leaving group or (CH₂)ₙ-LG wherein LG represents a suitable leaving group (such as chloro, bromo, iodo, mesylate, tosylate, nosylate or triflate) and p is 1 or 2.

Intermediates of type (VIII) (or its counterparts VIII-P and VIII-LG) can be obtained from compounds of formula (II) (or its counterparts II-P and II-LG) and reagents of formula (IXa-c) using the same reaction conditions as described above for methods A and B.

The reaction of an intermediate of general formula (VIII) (or its counterparts VIII-P and VIII-LG) to give a compound of formula (I) (or its counterparts V/VI-P and V/VI-LG, respectively) may be carried out under different reaction conditions, depending on the nature of the groups A and Z—W:
When A is an aldehyde and W is —(CH₂)ₚ—, by reductive amination reaction in the presence of a reductive reagent, preferably sodium triacetoxyborohydride, preferably in the presence of a base, preferably N,N-diisopropylethylamine (DIPEA) or triethylamine (TEA), in an organic solvent, preferably 1,2-dichloroethane (DCE).
When A is a carboxylic acid and W is —C(O)—, in the presence of a carboxylic acid activating reagent, preferably HATU (2-(7-Aza-1H-benzotriazole-1-yl)-1,1,3,3-tetramethyluronium) or EDCI (N-(3-Dimethylaminopropyl)-N′-ethylcarbodiimide hydrochloride), preferably in the presence of a base, preferably DIPEA or TEA, in an organic solvent, preferably dichloromethane (DCM). Alternatively, by conversion to the acid chloride intermediate using any suitable method.
When A is a good leaving group as a halogen atom and W is a bond, using a metal catalysed coupling, for example in the presence of a copper salt as catalyst, preferably CuI, an appropriate ligand, preferably N1,N2-dimethylethane-1,2-diamine or proline, and an inorganic base, preferably K₃PO₄ or K₂CO₃ in an organic solvent, preferably 1,4-dioxane, N,N-dimethylformamide (DMF) or DMSO, at a temperature range of 80-130° C. Alternatively, in the presence of a Pd catalyst, preferably Pd₂(dba)₃ and a suitable ligand, preferably 2-dicyclohexylphosphino-2′,4′,6′-triisopropylbiphenyl (Xphos), In the presence of a base, preferably NaOtBu, in an organic solvent, preferably toluene or 1,4-dioxane, at a temperature range of 50-150° C.
When A is a —(CH₂)ₚ-LG group (where LG is a good leaving group as a halogen atom or sulfonate), and W is —(CH₂)ₚ—, the reaction may be carried out in the presence of a base, preferably NaH, DIPEA or TEA, in an organic solvent, preferably DMF or THF, at a suitable temperature, preferably in the range of 0-100° C. Alternatively, in the presence of tetrabutylammonium iodide (TBAI).

The compounds of formula (II), (II-P) and (II-LG) are commercially available or can be obtained by reduction of the corresponding ketones, preferably using a hydride source. In addition, the reduction can be performed under asymmetric conditions described in the literature to render chiral compounds of formula (II) in enantiopure form. As a way of example, the chiral reduction can be performed using a hydride source such as borane-tetrahydrofuran complex or borane-dimethyl sulfide complex, in the presence of a Corey-Bakshi-Shibata oxazaborolidine catalyst, in a suitable solvent such as tetrahydrofuran or toluene, at a suitable temperature, preferably comprised between 0° C. and room temperature.

The compounds of general formula (III), (VII) and (IX) are commercially available or can be prepared by conventional methods described in the bibliography.

Some compounds of the present Invention can also be obtained starting from other compounds of general formula (I) by appropriate conversion reactions of functional groups, in one or several steps, using well-known reactions in organic chemistry under conventional experimental conditions.

In addition, a compound of general formula (I) can be obtained in enantiopure form by resolution of its corresponding racemic compound either by chiral preparative HPLC or by crystallization of a diastereomeric salt or co-crystal.

Alternatively, the resolution step can be carried out at a previous stage, using any suitable intermediate.

The obtained reaction products may, if desired, be purified by conventional methods, such as crystallization and chromatography. Where the processes described below for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

Another aspect of the invention refers to the process for obtaining the compounds [54] and [55] using the method A described above.

Turning to another aspect, the invention also relates to the therapeutic use of the compounds of general formula (I). As mentioned above, compounds of general formula (I) show a strong affinity to the subunit α2δ and more preferably to the α2δ-1 subunit of voltage-gated calcium channels. In a more preferred embodiment of the invention compounds of general formula (I) show a strong affinity to both the subunit α2δ and more preferably to the α2δ-1 subunit of voltage-gated calcium channels as well as the μ-opioid receptor (MOR or mu-opioid receptor) and can behave as agonists, antagonists, inverse agonists, partial antagonists or partial agonists thereof. Therefore, compounds of general formula (I) are useful as medicaments.

They are suitable for the treatment and/or prophylaxis of diseases and/or disorders mediated by the subunit α2δ, especially the α2δ-1 subunit of voltage-gated calcium channels and/or the μ-opioid receptor (MOR or mu-opioid receptor). In this sense, compounds of formula (I) are suitable for the treatment and/or prophylaxis of pain, especially neuropathic pain, inflammatory pain, and chronic pain or other pain conditions involving allodynia and/or hyperalgesia, depression, anxiety and attention-deficit-/hyperactivity disorder (ADHD).

The compounds of formula (I) are especially suited for the treatment of pain, especially neuropathic pain, inflammatory pain or other pain conditions involving allodynia and/or hyperalgesia. PAIN is defined by the International Association for the Study of Pain (IASP) as “an unpleasant sensory and emotional experience associated with actual or potential tissue damage, or described in terms of such damage (IASP, Classification of chronic pain, 2nd Edition, IASP Press (2002), 210). Even though pain is always subjective its causes or syndromes can be classified.

In a preferred embodiment compounds of the invention are used for the treatment and/or prophylaxis of allodynia and more specifically mechanical or thermal allodynia.

In another preferred embodiment compounds of the invention are used for the treatment and/or prophylaxis of hyperalgesia.

In yet another preferred embodiment compounds of the invention are used for the treatment and/or prophylaxis of neuropathic pain and more specifically for the treatment and/or prophylaxis of hyperpathia.

A related aspect of the invention refers to the use of compounds of formula (I) for the manufacture of a medicament for the treatment and/or prophylaxis of disorders and diseases mediated by the subunit α2δ, especially the α2δ-1 subunit of voltage-gated calcium channels and/or the μ-opioid receptor (MOR or mu-opioid receptor), as explained before.

Another related aspect of the Invention refers to a method for the treatment and/or prophylaxis of disorders and diseases mediated by the subunit α2δ, especially the α2δ-1 subunit of voltage-gated calcium channels and/or the μ-opioid receptor (MOR or mu-oploid receptor), as explained before comprising the administration of a therapeutically effective amount of a compound of general formula (I) to a subject in need thereof.

Another aspect of the invention is a pharmaceutical composition, which comprises at least a compound of general formula (I) or a pharmaceutically acceptable salt, prodrug, isomer or solvate thereof, and at least a pharmaceutically acceptable carrier, additive, adjuvant or vehicle.

The pharmaceutical composition of the invention can be formulated as a medicament in different pharmaceutical forms comprising at least a compound binding to the subunit α2δ, especially the α2δ-1 subunit of voltage-gated calcium channels and/or to the μ-opioid receptor (MOR or mu-opioid receptor) and optionally at least one further active substance and/or optionally at least one auxiliary substance.

The auxiliary substances or additives can be selected among carriers, excipients, support materials, lubricants, fillers, solvents, diluents, colorants, flavour conditioners such as sugars, antioxidants and/or agglutinants. In the case of suppositories, this may imply waxes or fatty acid esters or preservatives, emulsifiers and/or carriers for parenteral application. The selection of these auxiliary materials and/or additives and the amounts to be used will depend on the form of application of the pharmaceutical composition.

The pharmaceutical composition in accordance with the invention can be adapted to any form of administration, be it orally or parenterally, for example pulmonarily, nasally, rectally and/or intravenously.

Preferably, the composition is suitable for oral or parenteral administration, more preferably for oral, intravenous, intraperitoneal, intramuscular, subcutaneous, intrathekal, rectal, transdermal, transmucosal or nasal administration.

The composition of the invention can be formulated for oral administration in any form preferably selected from the group consisting of tablets, drageés, capsules, pills, chewing gums, powders, drops, gels, juices, syrups, solutions and suspensions. The composition of the present invention for oral administration may also be in the form of multiparticulates, preferably microparticles, microtablets, pellets or granules, optionally compressed into a tablet, filled into a capsule or suspended in a suitable liquid. Suitable liquids are known to those skilled in the art.

Suitable preparations for parenteral applications are solutions, suspensions, reconstitutable dry preparations or sprays.

The compounds of the Invention can be formulated as deposits in dissolved form or in patches, for percutaneous application.

In a preferred embodiment, the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

The daily dosage for humans and animals may vary depending on factors that have their basis in the respective species or other factors, such as age, sex, weight or degree of illness and so forth. The daily dosage for humans may preferably be in the range from 1 to 2000, preferably 1 to 1500, more preferably 1 to 1000 milligrams of active substance to be administered during one or several intakes per day.

The following examples are merely illustrative of certain embodiments of the invention and cannot be considered as restricting it in any way.

### EXAMPLES

In the next preparation examples the preparation of both intermediates compounds as well as compounds according to the invention are disclosed.

The following abbreviations are used:
ACN: Acetonitrile
Anh: Anhydrous
Aq: Aqueous
Cone: Concentration
CH: Cyclohexane
DCM: Dichloromethane
DCE: 1,2-Dichloroethane
DEA: Diethylamine
DIAD: Diisopropyl azodicarboxylate
DIBAL: Diisobutylaluminum hydride
DIPEA: N,N-Disopropylethylamine
DMA: N,N-Dimethylacetamide
DMSO: Dimethylsulfoxide
EtOAc: Ethyl acetate
EtOH: Ethanol
Ex: Example
h: Hour/s
HATU: 2-(7-Aza-1H-benzotriazole-1-yl)-1,1,3,3-tetramethyluronium hexafluorophosphate
Hex: Hexane
HPLC: High-performance liquid chromatography
INT: Intermediate
IPA: Isopropanol
MeOH: Methanol
MS: Mass spectrometry
Min: Minutes
Quant: Quantitative
Ret: Retention
rt: Room temperature
Sat: Saturated
TBAF: Tetrabutylammonium fluoride
TBAI: Tetrabutylammonium iodide
TEA: Triethylamine
TFA: Trifluoroacetic acid
THF: Tetrahydrofuran
XPhos: 2-Dicyclohexylphosphino-2′,4′,6′-triisopropylbiphenyl
Wt: Weight

The following methods were used to generate the HPLC or HPLC-MS data:

Method A: Column Eclipse XDB-C18 4.6×150 mm, 5 μm; flow rate 1 mL/min; A: H₂O (0.05% TFA); B: ACN; Gradient: 5% to 95% B In 7 min, isocratic 95% B 5 min.

Method B: Column Zorbax SB-C18 2.1×50 mm, 1.8 μm; flow rate 0.5 mL/min; A: H₂O (0.1% formic acid); 8: ACN (0.1% formic acid); Gradient: 5% to 95% 8 in 4 min, isocratic 95% B 4 min.

### Example 1: 3-(3-((3,5-Dimethylpiperazin-1-yl)methyl)phenoxy)-N-methyl-3-(thiophen-2-yl)propan-1-amine

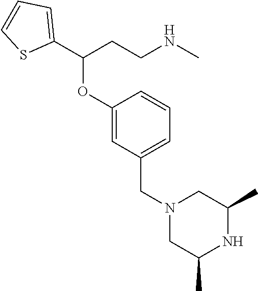 [SMILES: CNCCC(Oc1cccc(CN2C[C@@H](C)N[C@@H](C)C2)c1)c1cccs1]

### a) 3-(3-Chloro-1-(thiophen-2-yl)propoxy)benzaldehyde

To a solution of 3-chloro-1-(thiophen-2-yl)propan-1-ol (1.00 g, 5.66 mmol) in THF (10 mL) 3-hydroxybenzaldehyde (0.69 g, 5.66 mmol) and PPh₃ (1.63 g, 6.23 mmol) were added. The mixture was cooled to 0° C. and then DIAD (1.26 g, 6.23 mmol) was added dropwise. The reaction mixture was warmed slowly at rt and stirred for 16 h. The solvent was removed under vacuum and the residue was purified by flash chromatography, silica gel, gradient CH to 100% EtOAc to afford the title product (700 mg, 44% yield). HPLC (Method 8): Rot, 5.56 min; ESI⁺-MS m/z, 281.2 (M+H).

### b) 1-(3-(3-Chloro-1-(thiophen-2-yl)propoxy)benzyl)-3,5-dimethylpiperazine

To a solution of the compound obtained in step a (120 mg, 0.42 mmol) in DCE (2 mL), DIPEA (166 mg, 1.28 mmol), cis-2,6-dimethylpiperazine (122 mg, 1.06 mmol) and NaBH(OAc)₃ (181 mg, 0.85 mmol) were added and the mixture was stirred at rt for 16 h. NaHCO₃ sat solution was added, extracted with DCM and the organic layer was concentrated under vacuum. Purification by flash chromatography, silica gel, gradient Hex to 100% acetone afforded the title product (126 mg, 78% yield). HPLC (Method 8): Ret. 4.10 min: ES-MS m/z, 379.1 (M+H).

### c) Title Compound

To a solution of the compound obtained in step b (60 mg, 0.15 mmol) In EtOH (0.1 mL), methylamine (40% water solution, 0.41 mL, 4.75 mmol) was added and the mixture was heated in a sealed tube at 100° C. for 1 h. The mixture was cooled at rt and concentrated under vacuum. Purification by flash chromatography, silica gel, gradient DCM to 40% MeOH afforded the title product (36 mg, 55% yield). HPLC (Method A): Ret, 3.96 min: ESI⁺-MS m/z, 374.2 (M+H).

This method was used for the preparation of Ex 2-6 using suitable starting materials:

| | | | | Ret | |
|---|---|---|---|---|---|
| EX | Structure | Chemical name | Method | (min) | MS |
| 2 | 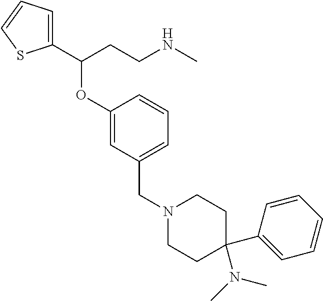 [SMILES: CNCCC(Oc1cccc(CN2CCC(c3ccccc3)(N(C)C)CC2)c1)c1cccs1] | N,N-dimethyl-1-(3-(3- (methylamino)-1- (thiophen-2- yl)propoxy)benzyl)-4- phenylpiperidin-4-amine | A | 4.14 | 464.3 (M + H) |
| 3 | 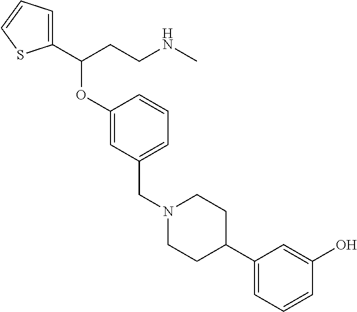 [SMILES: CNCCC(Oc1cccc(CN2CCC(c3cccc(O)c3)CC2)c1)c1cccs1] | 3-(1-(3-(3- (Methylamino)-1- (thiophen-2- yl)propoxy)benzyl)piper- idin-4-yl)phenol | A | 4.77 | 437.3 (M + H) |
| 4 | 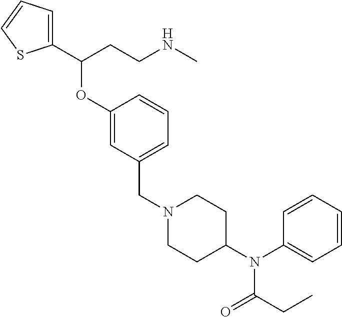 [SMILES: CCC(=O)N(c1ccccc1)C1CCN(Cc2cccc(OC(CCNC)c3cccs3)c2)CC1] | N-(1-(3-(3- (methylamino)-1- (thiophen-2- yl)propoxy)benzyl)piper- idin-4-yl)-N- phenylpropionamide | A | 4.99 | 492.3 (M + H) |
| 5 | 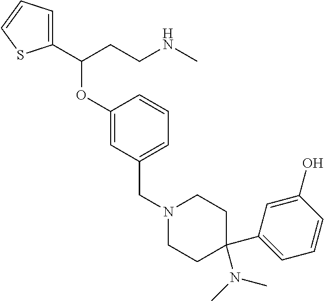 [SMILES: CNCCC(Oc1cccc(CN2CCC(c3cccc(O)c3)(N(C)C)CC2)c1)c1cccs1] | 3-(4-(Dimethylamino)-1- (3-(3-(methylamino)-1- (thiophen-2- yl)propoxy)benzyl)piper- idin-4-yl)phenol | A | 4.05 | 480.3 (M + H) |
| 6 | 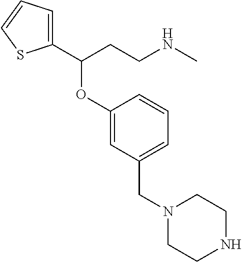 [SMILES: CNCCC(Oc1cccc(CN2CCNCC2)c1)c1cccs1] | N-methyl-3-(3- (piperazin-1- ylmethyl)phenoxy)-3- (thiophen-2-yl)propan-1- amine | A | 3.81 | 346.2 (M + H) |

### Example 7: (4-(Dimethylamino)-4-phenylpiperidin-1-yl)(3-(3-(methylamino)-1-(thiophen-2-yl)propoxy)phenyl)methanone

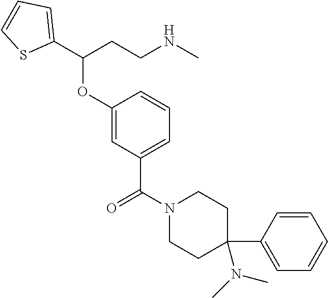 [SMILES: CNCCC(Oc1cccc(C(=O)N2CCC(c3ccccc3)(N(C)C)CC2)c1)c1cccs1]

### a) Methyl 3-(3-chloro-1-(thiophen-2-yl)propoxy)benzoate

3-Chloro-1-(thiophen-2-yl)propan-1-ol was treated with methy 3-hydroxybenzoate in the conditions used in EX 1 step a), to afford the title compound (34% yield). HPLC (Method B): Ret, 5.80 min; ESI--MS m/z, 309.1 (M−H).

### b) 3-(3-Chloro-1-(thiophen-2-yl)propoxy)benzoic Acid

To a solution of the compound obtained in step a) (360 mg, 1.15 mmol) in a (1:1) mixture of THF and water (12 mL), LiOH (166 mg, 6.95 mmol) was added and the mixture was heated at 100° C. for 1 h. The reaction mixture was cooled at rt, citric acid solution was added until pH=5 and extracted with DCM to afford the title compound that was used in the next step without further purification (quant yield). HPLC (Method B): Ret, 5.14 min; ESI⁺-MS m/z, 319.0 (M+Na).

### c) (3-(3-Chloro-1-(thiophen-2-yl)propoxy)phenyl)(4-(dimethylamino)-4-phenylpiperidin-1-yl)methanone

To a solution of the compound obtained in step b) (93 mg, 0.33 mmol) in DCM (5 mL), HATU (0.128 g, 0.33 mmol) was added and the mixture was stirred at rt for 30 min. DIPEA (131 mg, 1.01 mmol) and N,N-dimethyl-4-phenylpiperidin-4-amine dihydrochloride (93 mg, 0.33 mmol) were added and the mixture was stirred at rt for 16 h. DCM was added, washed with water and brine, dried with Na₂SO₄ and the solvent was removed under vacuum. Purification by flash chromatography, silica gel, gradient Hex to 100% EtOAc afforded the title product (111 mg, 68% yield). HPLC (Method B): Ret 4.11 min; ESI⁺-MS m/z, 483.2 (M+H).

### d) Title Compound

The compound obtained in step c) was treated with the conditions used in EX 1 step c) to afford the title compound (47% yield). HPLC (Method A): Ret, 4.68 min; ESI⁺-MS m/z, 478.3 (M+H).

This method was used for the preparation of Ex 8-11 using suitable starting materials:

| | | | | Ret | |
|---|---|---|---|---|---|
| EX | Structure | Chemical name | Method | (min) | MS |
| 8 | 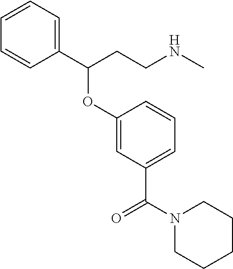 [SMILES: CNCCC(Oc1cccc(C(=O)N2CCCCC2)c1)c1ccccc1] | (3-(3-(Methylamino)-1- phenylpropoxy)phenyl)- (piperidin-1-yl)methanone | A | 5.15 | 353.2 (M + H) |
| 9 | 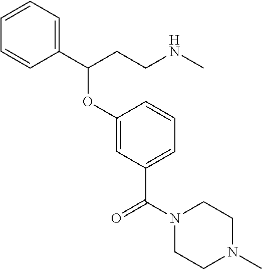 [SMILES: CNCCC(Oc1cccc(C(=O)N2CCN(C)CC2)c1)c1ccccc1] | (3-(3-(Methylamino)-1- phenylpropoxy)phenyl)(4- methylpiperazin-1- yl)methanone | A | 4.16 | 368.2 (M + H) |
| 10 | 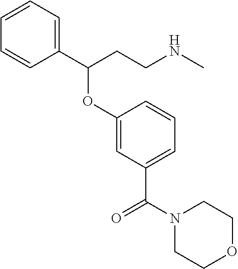 [SMILES: CNCCC(Oc1cccc(C(=O)N2CCOCC2)c1)c1ccccc1] | (3-(3-(Methylamino)-1- phenylpropoxy)phenyl- (morpholino)methanone | A | 5.02 | 355.2 (M + H) |
| 11 | 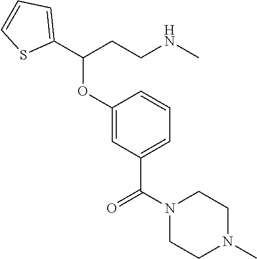 [SMILES: CNCCC(Oc1cccc(C(=O)N2CCN(C)CC2)c1)c1cccs1] | (3-(3-(Methylamino)-1- (thiophen-2- yl)propoxy)phenyl)(4- methylpiperazin-1- yl)methanone | A | 4.00 | 374.2 (M + H) |

### Example 12: (3R)-3-(3-(2-(4-(4-ethyl-2,2-dimethyltetrahydro-2H-pyran-4-yl)piperidin-1-yl)ethyl)phenoxy)-N-methyl-3-phenylpropan-1-amine

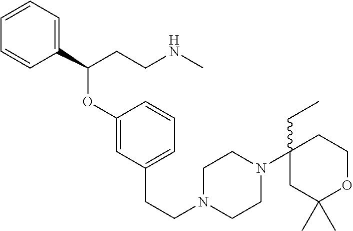 [SMILES: CCC1(N2CCN(CCc3cccc(O[C@H](CCNC)c4ccccc4)c3)CC2)CCOC(C)(C)C1]

### a) (R)-tert-butyl(3-(3-chloro-1-phenylpropoxy)phenethoxy)dimethylsilane

(R)-3-Chloro-1-phenylpropan-1-ol was treated with 3-(2-((tert-butyldimethylsilyl)oxy) ethyl)phenol in the conditions used in Ex 1 step a), to afford the title compound (63% yield). ESI⁺-MS m/z, 4272 (M+Na).

### b) (R)-3(3-(2-((tert-butyldimethylsilyl)oxy)ethyl)phenoxy)-N-methyl-3-phenylpropen-amine

The compound obtained in step a) was treated with the conditions used in Ex 1 step c) to afford the title compound that was used in the next step without further purification.

### c) tert-Butyl (R)-3-(3(2-(tert-butyldimethylsilyl)oxy)ethyl)phenoxy)-3-phenylpropyl) (methyl)carbamate

To a solution of the compound obtained in step b (469 mg, 1.17 mmol) in DCM (24 mL) cooled at 0° C., di-tert-butyldicarbonate (282 mg, 1.29 mmol) was added and the reaction mixture was stirred at rt for 16 h. Water was added, the mixture was extracted with DCM and washed with NaHCO₃ sat solution and brine. The solvent was removed under vacuum. Purification by flash chromatography, silica gel, gradient CH to 50% EtOAc, afforded the title compound (537 mg, 92% yield). HPLC (Method B): Ret, 8.60 min; ESI⁺-MS m/z, 522.3 (M+Na).

### d) tert-Butyl (R)-(3-(3-(2-hydroxyethyl)phenoxy)-3-phenylpropyl)(methyl)carbamate

To a solution of the compound obtained in step c) (527 mg, 1.05 mmol) In THF (5 mL), TBAF (1M solution in THF, 1.58 mL, 1.58 mmol) was added and the mixture was stirred at rt for 2 h. The reaction mixture was concentrated under vacuum. Purification by flash chromatography, silica gel, gradient CH to 100% EtOAc, afforded the title compound (400 mg, 95% yield). HPLC (Method B): Ret, 5.49 min; ESI⁺-MS m/z, 408.2 (M+Na).

### e) tert-Butyl (R)-methyl(3-(3-(2-oxoethyl)phenoxy)-3-phenylpropyl)carbamat

To a mixture of dry DMSO (0.1 mL) and dry DCM (5 mL) cooled at −78° C., oxalyl chloride (0.53 mL, 1.06 mmol) was added and the mixture was stirred for 15 min. A solution of the compound obtained in step d) (227 mg, 0.59 mmol) in dry DCM (2.5 mL) was dropwise added and the mixture was stirred at −78° C. for 40 min. DIPEA (0.51 mL, 2.94 mmol) was added and the reaction mixture was stirred at −78° C. for 10 min and then at 0° C. for 20 min. NH₄Cl sat solution was added, extracted with DCM and concentrated under vacuum. Purification by flash chromatography, silica gel, gradient Hex to 100% EtOAc, afforded the title compound (109 mg, 39% yield). ¹H-NMR (CDCl₃, 300 MHz) δ (ppm): 9.66 (m, 1H), 7.39-7.14 (m, 6H), 6.73 (m, 3H), 5.13 (m, 1H), 3.57 (m, 2H), 3.44 (m, 2H), 2.85 (s, 3H), 2.12 (m, 2H), 1.40 (bs, 9H).

### f) tert-Butyl ((3R)-3-(3-(2(4-(4-ethyl-2,2-dimethyltetrahydro-2H-pyran-4-yl)piperazin-1-yl)ethyl)phenoxy)-3-phenylpropyl)(methyl)carbamate

The compound obtained in step e) was treated with 1-(4-ethyl-2,2-dimethyltetrahydro-2H-pyran-4-yl)piperazine in the conditions used in Ex 1 step b) to afford the title compound (111 mg, 56% yield) as a mixture of two diastereomers. HPLC (Method A): Ret, 7.35 min; ESI⁺-MS m/z, 594.5 (M+H).

### g) Title Compound

To a solution of the compound obtained in step f) (96 mg, 0.18 mmol, as a mixture of two diastereomers) in dioxane (0.40 mL), HCl (4M solution in dioxane, 0.566 mL, 2.26 mmol) was added and the mixture was stirred at rt for 1 h. The reaction mixture was concentrated to dryness under vacuum. To obtain the free base, DCM was added, washed with Na₂CO₃ (10% solution), and the aqueous phase was extracted with DCM. The combined organic layers were concentrated under vacuum to afford the title compound (80 mg, quant) as a mixture of two diasteromers. HPLC (Method A): Ret, 5.02 min; ESI⁺-MS m/h, 494.3 (M+H).

The two diastereomers were separated by semipreparative HPLC. Conditions: column Chiralpak IB 250×10 mm; mobile phase, isocratic Hex:IPA:DEA (90:10:0.4); flux 5 ml/min; conc. 10 mg/mL; Ret 8.4 (Ex 12a) and 10.9 min (Ex 12b).

### Example 13: 3-(3-(3,5-Dimethyl-1H-pyrazol-1-yl)phenoxy)-N-methyl-3-phenyl propen-1-amine

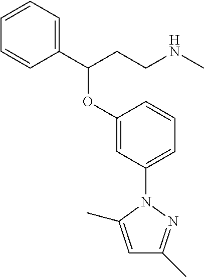 [SMILES: CNCCC(Oc1cccc(-n2nc(C)cc2C)c1)c1ccccc1]

### a) 1-(3-(3-Chloro-1-phenylpropoxy)phenyl)-3,5-dimethyl-1H-pyrazole

3-Chloro-1-phenylpropan-1-ol was treated with 3-(3,5-dimethyl-1H-pyrazol-1-yl)phenol in the conditions used in Ex 1 step a to afford the title compound that was used in the next step without further purification.

### b) Title Compound

The compound obtained in step a) was treated with the conditions used in Ex 1 step c) to afford the title compound (40% yield). HPLC-MS (Method A): Ret, 5.82 min; ESI⁺-MS m/z, 336.2 (M+H).

### Example 14: (S)-3-(4-(dimethylamino)-1-(3-(3-(methylamino)-1-(thiophen-2-yl) propoxy)benzyl)piperidin-4-yl)phenol

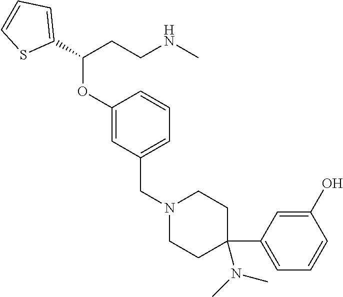 [SMILES: CNCC[C@H](Oc1cccc(CN2CCC(c3cccc(O)c3)(N(C)C)CC2)c1)c1cccs1]

### a) (S)-3-(3-(methylamino)-1-(thiophen-2-yl)propoxy)benzonitrile

To a solution of (S) 3-(methylamino)-1-(thiophen-2-yl)propan-ol (300 mg, 1.75 mmol) in DMA (3 mL), NaH (105 mg, 60% suspension in mineral oil, 2.63 mmol) was added and the solution was stirred at rt for 30 min. A solution of 3-fluorobenzonitrile (318 mg, 2.63 mmol) in DMA (2 mL) was added and the mixture was heated at 90° C. for 3 h. Water was added and extracted with EtOAc. The organic layer was dried with Na₂SO₄ and the solvent was removed under vacuum to afford the title compound that was used in the next step without further purification.

### b) 2-(Trimethylsilyl)ethyl (S)(3-(3-cyanophenoxy)-3-(thiophen-2-yl)propyl)(methyl) carbamate

To a solution of the compound obtained in step a) (500 mg, 1.84 mmol) in DCM (6 mL), DIPEA (0.32 mL, 1.84 mmol) and a solution of 4-nitrophenyl (2-(trimethylsilyl)ethyl)carbonate (520 mg, 1.84 mmol) in DCM (6 mL) were added and the reaction mixture was stirred at rt for 16 h. The reaction mixture was washed with NaHCO₃ sat solution and then with 2 M NaOH aq solution (three times). The organic layer was dried with Na₂SO₄, the solvent was removed under vacuum and the residue was purified by flash chromatography, silica gel, gradient CH to 100% EtOAc, to afford the title compound (600 mg, 78% global yield, 2 steps). HPLC-MS (Method 8): Ret, 6.29 min; ESI⁺-MS m/z, 439.1.

### c) 2-(Trimethylsilyl)ethyl (S)-(3-(3-formylphenoxy)-3-(thiophen-2-yl)propyl)(methyl) carbamate

To a solution of the compound obtained in step b) (550 mg, 1.32 mmol) in toluene (10 mL) at 0° C. under Ar atmosphere. DIBAL (1 M solution in toluene, 1.58 mL, 1.58 mmol) was dropwise added and the mixture was stirred at 0° C. for additional 2 h. HCl 10% aq solution was added at 0° C. and the mixture was stirred at rt for 1 h. The aq layer was extracted with DCM, the organic layer was washed with water and brine and the solvent was removed under vacuum. Purification by flash chromatography, silica gel, gradient CH to 100% EtOAc, afforded the title compound (172 mg, 30% yield). HPLC-MS (Method 8): Ret, 6.23 min; ESI⁺-MS m/z, 442.1 (M+Na).

### d) 2-(Trimethylsilyl)ethyl (S)-(3-(3-((4-(dimethylamino)-4-(3-hydroxyphenyl)piperidin-1-yl)methyl)phenoxy)-3-(thiophen-2-yl)propyl)(methyl)carbamate

The compound obtained in step c) (160 mg, 0.38 mmol) was treated with 3-(4-(dimethylamino)piperidin-4-yl)phenol dihydrochloride (134 mg, 0.45 mmol) with the conditions used in Ex 1 step b) to afford the title compound (62 mg, 26% yield). HPLC-MS (Method B): Ret, 4.27 min; ESI⁺-MS m/z, 624.3 (M+H).

### e) Title Compound

To a solution of the compound obtained in step d) (58 mg, 0.09 mmol) in DMF (2 mL), CsF (71 mg, 0.46 mmol) was added and the mixture was heated at 90° C. for 1.5 h. The reaction mixture was cooled to rt and the solvent was removed under vacuum. Purification by flash chromatography, silica gel, gradient DCM to 40% MeOH, afforded the title compound (34 mg, 76% yield). HPLC-MS (Method A): Ret, 4.05 min: ESI⁺-MS m/, 480.3 (M+H).

This method was used for the preparation of Ex 15-31 using suitable starting materials:

| | | | | Ret | |
|---|---|---|---|---|---|
| EX | Structure | Chemical name | Method | (min) | MS |
| 15 | 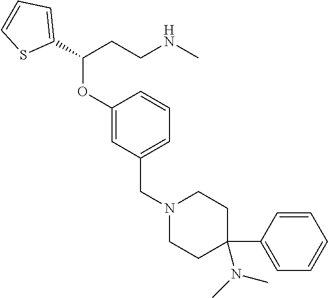 [SMILES: CNCC[C@H](Oc1cccc(CN2CCC(c3ccccc3)(N(C)C)CC2)c1)c1cccs1] | (S)-N,N-dimethyl-1-(3- (3-(methylamino)-1- (thiophen-2- yl)propoxy)benzyl)-4- phenylpiperidin-4- amine | A | 4.16 | 464.3 (M + H) |
| 16 | 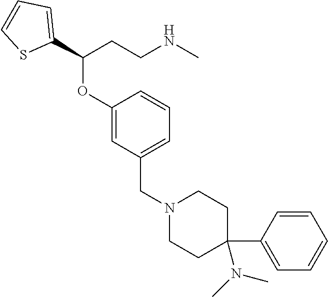 [SMILES: CNCC[C@@H](Oc1cccc(CN2CCC(c3ccccc3)(N(C)C)CC2)c1)c1cccs1] | (R)-N,N-dimethyl-1-(3- (3-(methylamino)-1- (thiophen-2- yl)propoxy)benzyl)-4- phenylpiperidin-4- amine | A | 4.16 | 464.3 (M + H) |
| 17 | 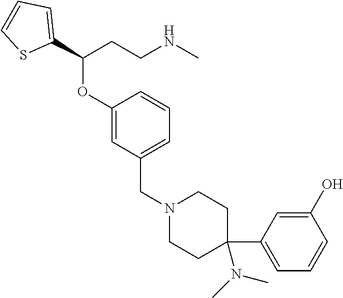 [SMILES: CNCC[C@@H](Oc1cccc(CN2CCC(c3cccc(O)c3)(N(C)C)CC2)c1)c1cccs1] | (R)-3-(4- (dimethylamino)-1-(3- (3-(methylamino)-1- (thiophen-2- yl)propoxy)benzyl)piper- idin-4-yl)phenol | A | 4.05 | 480.3 (M + H) |
| 18 | 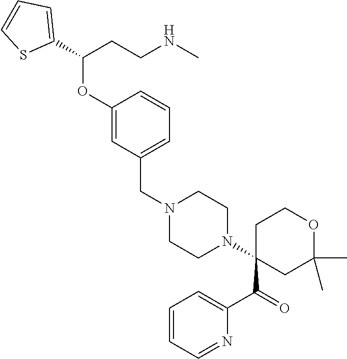 [SMILES: CNCC[C@H](Oc1cccc(CN2CCN([C@]3(C(=O)c4ccccn4)CCOC(C)(C)C3)CC2)c1)c1cccs1] | ((R)-2,2-dimethyl-4-(4- (3-((S)-3- (methylamino)-1- (thiophen-2- yl)propoxy)benzyl)piper- azin-1-yl)tetrahyrdro- 2H-pyran-4-yl)(pyridin- 2-yl)methanone | A | 5.11 | 563.3 (M + H) |
| 19 | 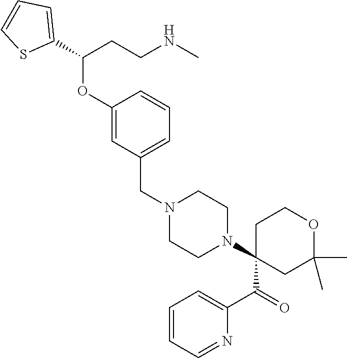 [SMILES: CNCC[C@H](Oc1cccc(CN2CCN([C@@]3(C(=O)c4ccccn4)CCOC(C)(C)C3)CC2)c1)c1cccs1] | ((S)-2,2-dimethyl-4-(4- (3-((S)-3- (methylamino)-1- (thiophen-2- yl)propoxy)benzyl)piper- azin-1-yl)tetrahydro- 2H-pyran-4-yl)(pyridin- 2-yl)methanone | A | 5.11 | 563.3 (M + H) |
| 20 | 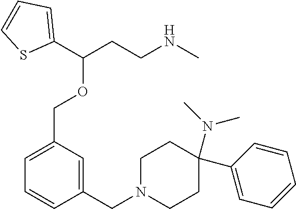 [SMILES: CNCCC(OCc1cccc(CN2CCC(c3ccccc3)(N(C)C)CC2)c1)c1cccs1] | N,N-dimethyl-1-(3-((3- (methylamino)-1- (thiophen-2- yl)propoxy)methyl)ben- zyl)-4-phenylpiperidin- 4-amine | A | 4.26 | 478.3 (M + H) |
| 21 | 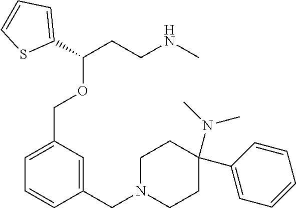 [SMILES: CNCC[C@H](OCc1cccc(CN2CCC(c3ccccc3)(N(C)C)CC2)c1)c1cccs1] | (S)-N,N-dimethyl-1-(3- ((3-(methylamino)-1- (thiophen-2- yl)propoxy)methyl)ben- zyl)-4-phenylpiperidin- 4-amine | A | 4.26 | 478.3 (M + H) |
| 22 | 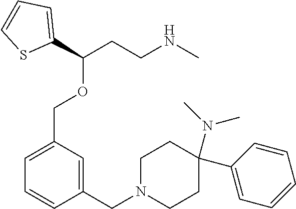 [SMILES: CNCC[C@@H](OCc1cccc(CN2CCC(c3ccccc3)(N(C)C)CC2)c1)c1cccs1] | (R)-N,N-dimethyl-1-(3- ((3-(methylamino)-1- (thiophen-2- yl)propoxy)methyl)ben- zyl)-4-phenylpiperidin- 4-amine | A | 4.26 | 478.3 (M + H) |
| 23 | 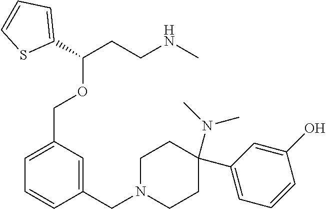 [SMILES: CNCC[C@H](OCc1cccc(CN2CCC(c3cccc(O)c3)(N(C)C)CC2)c1)c1cccs1] | (S)-3-(4- (dimethylamino)-1-(3- ((3-(methylamino)-1- (thiophen-2- yl)propoxy)methyl)ben- zyl)piperidin-4- yl)phenol | A | 4.18 | 494.3 (M + H) |
| 24 | 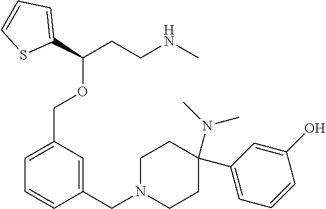 [SMILES: CNCC[C@@H](OCc1cccc(CN2CCC(c3cccc(O)c3)(N(C)C)CC2)c1)c1cccs1] | (R)-3-(4- (dimethylamino)-1-(3- ((3-(methylamino)-1- (thiophen-2- yl)propoxy)methyl)ben- zyl)piperidin-4- yl)phenol | A | 4.18 | 494.3 (M + H) |
| 25 | 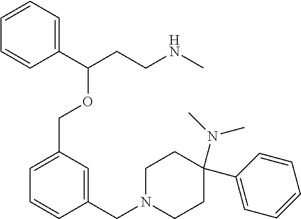 [SMILES: CNCCC(OCc1cccc(CN2CCC(c3ccccc3)(N(C)C)CC2)c1)c1ccccc1] | N,N-dimethyl-1-(3-((3- ((methylamino)-1- phenylpropoxy)methyl) benzyl)-4- phenylpiperidin-4- amine | A | 4.41 | 472.3 (M + H) |
| 26 | 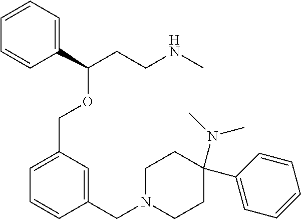 [SMILES: CNCC[C@@H](OCc1cccc(CN2CCC(c3ccccc3)(N(C)C)CC2)c1)c1ccccc1] | (R)-N,N-dimethyl-1-(3- ((3-(methylamino)-1- phenylpropoxy)methyl) benzyl)-4- phenylpiperidin-4- amine | A | 4.41 | 472.3 (M + H) |
| 27 | 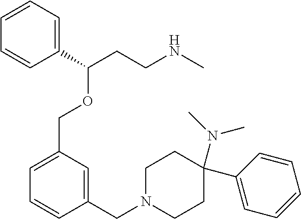 [SMILES: CNCC[C@H](OCc1cccc(CN2CCC(c3ccccc3)(N(C)C)CC2)c1)c1ccccc1] | (S)-N,N-dimethyl-1-(3- ((3-(methylamino)-1- phenylpropoxy)methyl) benzyl)-4- phenylpiperidin-4- amine | A | 4.41 | 472.3 (M + H) |
| 28 | 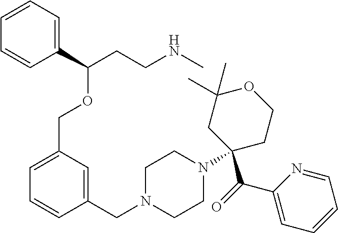 [SMILES: CNCC[C@@H](OCc1cccc(CN2CCN([C@@]3(C(=O)c4ccccn4)CCOC(C)(C)C3)CC2)c1)c1ccccc1] | ((S)-2,2-dimethyl-4-(4- (3-(((R)-3- (methylamino)-1- phenylpropoxy)methyl) benzyl)piperazin-1- yl)tetrahydro-2H- pyran-4-yl)(pyridin-2- yl)methanone | A | 5.33 | 571.4 (M + H) |
| 29 | 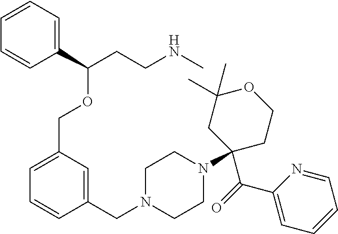 [SMILES: CNCC[C@@H](OCc1cccc(CN2CCN([C@]3(C(=O)c4ccccn4)CCOC(C)(C)C3)CC2)c1)c1ccccc1] | ((R)-2,2-dimethyl-4-(4- (3-(((R)-3- (methylamino)-1- phenylpropoxy)methyl) benzyl)piperazin-1- yl)tetrahydro-2H- pyran-4-yl)(pyridin-2- yl)methanone | A | 5.33 | 571.4 (M + H) |
| 30 | 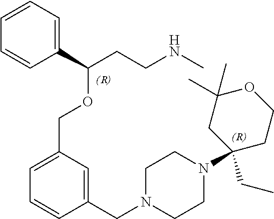 | (R)-3-((3-((4-((R)-4- ethyl-2,2- dimethyltetrahydro-2H- pyran-4-yl)piperazin-1- yl)methyl)benzyl)oxy)- N-methyl-3- phenylpropan-1-amine | A | 5.07 | 494.4 (M + H) |
| 31 | 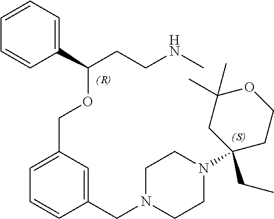 | (R)-3-((3-((4-((S)-4- ethyl-2,2- dimethyltetrahydro-2H- pyran-4-yl)piperazin-1- yl)methyl)benzyl)oxy)- N-methyl-3- phenylpropan-1-amine | A | 5.07 | 494.4 (M + H) |

In Ex 25-31 Boc was used as protect ng group.

### Example 32: 2-(3-((3-(Methylamino)-1-(thiophen-2-yl)propoxy)methyl)phenyl)-3,4-dihydroisoquinolin-1(2H)-one

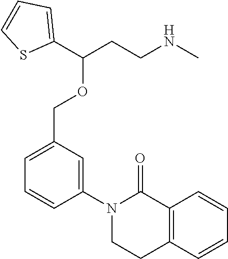 [SMILES: CNCCC(OCc1cccc(N2CCc3ccccc3C2=O)c1)c1cccs1]

### a) tert-Butyl (3-((3-bromobenzyl)oxy)-3-(thiophen-2-yl)propyl)(methyl)carbamate

To a solution of tert-butyl (3-hydroxy-3-(thiophen-2-yl)propyl)(methy)carbamate (600 mg, 2.21 mmol) in DMF (8 mL) cooled at 0° C., NaH (133 mg, 60% suspension in mineral oil, 3.33 mmol) was added and the solution was stirred at rt for 30 min. Then, the reaction mixture was cooled again at 0° C. and a solution of 1-bromo-3-(bromomethyl)benzene (829 mg, 3.32 mmol) in DMF (4 mL) was added. The reaction mixture was stirred at rt for 3 h, water was added carefully and extracted with EtOAc; the organic phase was dried with Na₂SO₄ and the solvent was removed under vacuum. Purification by flash chromatography, silica gel, gradient from CH to 100% EtOAc afforded the title product (925 mg, 95% yield). HPLC (Method B): Ret, 6.62 min; ESI⁺-MS m/z, 462.0 (M+Na).

### b) tert-Butyl methyl(3-((3-(1-oxo-3,4-dihydroisoquinolin-2(1H)-yl)benzyl)oxy)-3-(thiophen-2-yl)propyl)carbamate

A mixture of CuI (37 mg, 0.19 mmol) and N1,N2-dimethylethane-1,2-diamine (17 mg, 0.19 mmol) in dioxane (0.5 mL) was stirred at rt for 20 min. A solution of the compound obtained in step a) (150 mg, 0.34 mmol), 3,4-dihydroisoquinolin-1(2H)-one (55 mg, 0.37 mmol) and K₃PO₄ (145 mg, 0.68 mmol) were added and the mixture was heated at 130° C. under Ar atmosphere for 20 h. The reaction mixture was cooled to rt and the solvent was removed under vacuum. Purification by flash chromatography, silica gel, gradient Hex to 100% EtOAc afforded the title compound (150 mg, 87% yield). HPLC (Method B): Ret, 6.12 min; ESI⁺-MS m/z, 529.2 (M+Na).

### c) Title Compound

In a round bottomed flask, ZnBr₂ (333 mg, 1.48 mmol) was dried under vacuum at 240° C. for 3 h. Once the solid reached rt, a solution of the compound obtained in step b) (296 mg, 0.15 mmol) in DCM (10 mL) was added and the mixture was stirred at rt under Ar atmosphere for 20 h. Water was added and the mixture was stirred for 1 h. The phases were separated and the aqueous phase was extracted with DCM. The organic phase was concentrated under vacuum and purified by flash chromatography, silica gel, gradient DCM to 25% MeOH, to afford the title compound (96 mg, 79% yield). HPLC (Method A): Ret, 6.14 min: ESI⁺-HRMS m/z, 407.2 (M+H).

This method was used for the preparation of Ex 33-41 using suitable starting materials:

| | | | | Ret | |
|---|---|---|---|---|---|
| EX | Structure | Chemical name | Method | (min) | MS |
| 33 | 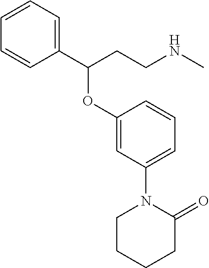 [SMILES: CNCCC(Oc1cccc(N2CCCCC2=O)c1)c1ccccc1] | 1-(3-(3-(Methylamino)- 1- phenylpropoxy)phenyl) piperidin-2-one | A | 5.38 | 339.2 (M + H) |
| 34 | 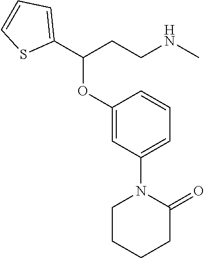 [SMILES: CNCCC(Oc1cccc(N2CCCCC2=O)c1)c1cccs1] | 1-(3-(3-(Methylamino)- 1-(thiophen-2- yl)propoxy)phenyl)piper- idin-2-one | A | 5.19 | 345.1 (M + H) |
| 35 | 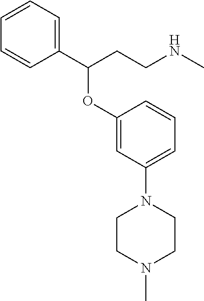 [SMILES: CNCCC(Oc1cccc(N2CCN(C)CC2)c1)c1ccccc1] | N-methyl-3-(3-(4- methylpiperazin-1- yl)phenoxy)-3- phenylpropan-1-amine | A | 4.42 | 340.2 (M + H) |
| 36 | 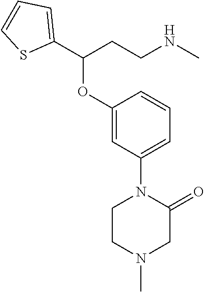 [SMILES: CNCCC(Oc1cccc(N2CCN(C)CC2=O)c1)c1cccs1] | 4-Methyl-1-(3-(3- (methylamino)-1- (thiophen-2- yl)propoxy)phenyl)piper- azin-2-one | A | 4.06 | 382.1 (M + H) |
| 37 | 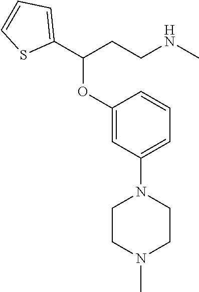 [SMILES: CNCCC(Oc1cccc(N2CCN(C)CC2)c1)c1cccs1] | N-methyl-3-(3-(4- methylpiperazin-1- yl)phenoxy)-3- (thiophen-2-yl)propan- 1-amine | A | 4.35 | 346.1 (M + H) |
| 38 | 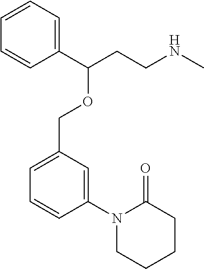 [SMILES: CNCCC(OCc1cccc(N2CCCCC2=O)c1)c1ccccc1] | 1-(3-((3- (Methylamino)-1- phenylpropoxy)methyl) phenyl)piperidin-2-one | A | 5.53 | 353.2 (M + H) |
| 39 | 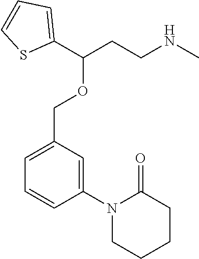 [SMILES: CNCCC(OCc1cccc(N2CCCCC2=O)c1)c1cccs1] | 1-(3-((3- (Methylamino)-1- (thiophen-2- yl)propoxy)methyl)phe- nyl)piperidin-2-one | A | 5.40 | 359.1 (M + H) |
| 40 | 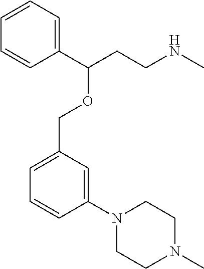 [SMILES: CNCCC(OCc1cccc(N2CCN(C)CC2)c1)c1ccccc1] | N-methyl-3-((3-(4- methylpiperazin-1- yl)benzyl)oxy)-3- phenylpropan-1-amine | A | 4.61 | 354.2 (M + H) |
| 41 | 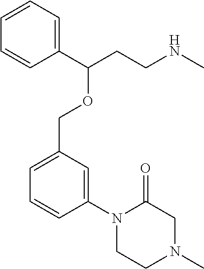 [SMILES: CNCCC(OCc1cccc(N2CCN(C)CC2=O)c1)c1ccccc1] | 4-Methyl-1-(3-((3- (methylamino)-1- phenylpropoxy)methyl) phenyl)piperazin-2-one | A | 4.39 | 368.2 (M + H) |

### Example 42: N-methyl-3-(3-(piperazin-1-yl)phenoxy)-3-(thiophen-2-yl)propan-1-amine

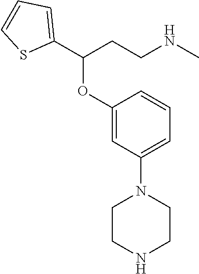 [SMILES: CNCCC(Oc1cccc(N2CCNCC2)c1)c1cccs1]

### a) 2-(Trimethylsilyl)ethyl (3-(3-iodophenoxy)-3-(thiophen-2-yl)propyl)(methyl) carbamate

3-(3-Iodophenoxy)-N-methyl-3-(thiophen-2-yl)propan-1-amine was treated with the conditions used in Ex 14 step b), to afford the title compound (68% yield). HPLC (Method B): Ret, 7.05 min; ESI⁺-HRMS m/z, 540.0 (M+Na).

### b) 2-(Trimethylsilyl)ethyl methyl(3-(3-yl)propyl) carbamate

A sealed tube was charged with Pd₂(dba)₃ (15 mg, 0.015 mmol), XPhos (30 mg, 0.06 mmol), potassium tert-butoxide (42 mg, 0.43 mmol) and piperazine (80 mg, 0.92 mmol) under Ar atmosphere. A solution of the compound obtained in step a) (160 mg, 0.31 mmol) in dioxane (2 mL) was added and the mixture was heated at 110° C. for 20 h. The reaction mixture was cooled at rt, filtered through a pad of celite and the solution was concentrated under vacuum. Purification by flash chromatography, silica gel, gradient DCM to 30% MeOH, afforded the title compound (78 mg, 63% yield). HPLC (Method B): Ret, 4.70 min; ESI⁺-MS m/z, 476.2 (M+H).

### c) Title Compound

The compound obtained in step b) was treated with the conditions used in Ex 14 step d), to afford the title compound (68% yield). HPLC (Method A): Ret, 4.25 min; ESI⁺-MS m/z, 332.2 (M+H).

This method was used for the preparation of Ex 43-49 using suitable starting materials:

| | | | | Ret | |
|---|---|---|---|---|---|
| EX | Structure | Chemical name | Method | (min) | MS |
| 43 | 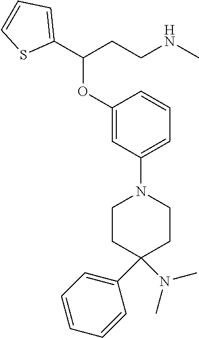 [SMILES: CNCCC(Oc1cccc(N2CCC(c3ccccc3)(N(C)C)CC2)c1)c1cccs1] | N,N-dimethyl-1-(3-(3- (methylamino)-1- (thiophen-2- yl)propoxy)phenyl)-4- phenylpiperidin-4- amine | A | 4.95 | 450.3 (M + H) |
| 44 | 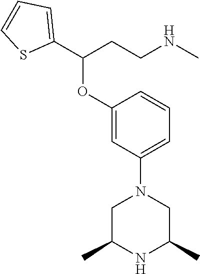 [SMILES: CNCCC(Oc1cccc(N2C[C@@H](C)N[C@@H](C)C2)c1)c1cccs1] | 3-(3-((3S,5R)-3,5- dimethylpiperazin-1- yl)phenoxy)-N-methyl- 3-(thiophen-2- yl)propan-1-amine | A | 4.41 | 360.2 (M + H) |
| 45 | 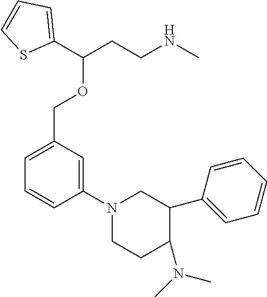 [SMILES: CNCCC(OCc1cccc(N2CCC(N(C)C)C(c3ccccc3)C2)c1)c1cccs1] | N,N-dimethyl-1-(3-((3- (methylamino)-1- (thiophen-2- yl)propoxy)methyl)phe- nyl)-4-phenylpiperidin- 4-amine | A | 5.10 | 464.3 (M + H) |
| 46 | 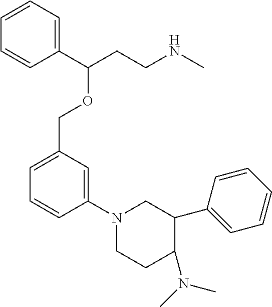 [SMILES: CNCCC(OCc1cccc(N2CCC(N(C)C)C(c3ccccc3)C2)c1)c1ccccc1] | N,N-dimethyl-1-(3-((3- (methylamino)-1- phenylpropoxy)methyl) phenyl)-4- phenylpiperidin-4- amine | A | 5.16 | 458.3 (M + H) |
| 47 | 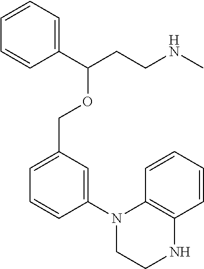 [SMILES: CNCCC(OCc1cccc(N2CCNc3ccccc32)c1)c1ccccc1] | 3-((3-(3,4- Dihydroquinoxalin- 1(2H)-yl)benzyl)oxy)- N-methyl-3- phenylpropan-1-amine | A | 5.41 | 388.2 (M + H) |
| 48 | 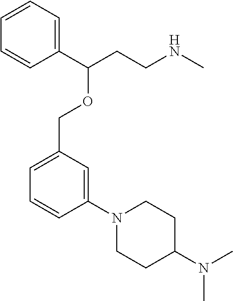 [SMILES: CNCCC(OCc1cccc(N2CCC(N(C)C)CC2)c1)c1ccccc1] | N,N-dimethyl-1-(3-((3- (methylamino)-1- phenylpropoxy)methyl) phenyl)piperidin-4- amine | A | 4.56 | 382.3 (M + H) |
| 49 | 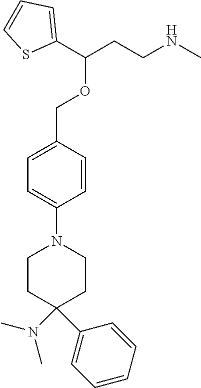 | N,N-dimethyl-1-(4-((3- (methylamino)-1- (thiophen-2- yl)propoxy)methyl)phe- nyl)-4-phenylpiperidin- 4-amine | A | 5.09 | 486.3 (M + H) |

### Example 50: (S)-(4-(3-(methylamino)-1-(thiophen-2-yl)propoxy)benzyl)-3,4-dihydroisoquinolin-1(2H)-one

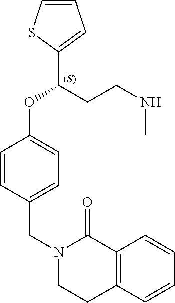

### a) 2-(Trimethylsilyl)ethyl (S)-methyl(3-(4-((1-oxo-3,4-dihydroisoquinolin-2(1H)-yl) methyl)phenoxy)-3-(thiophen-2-yl)propyl)carbamate

To a solution of 3,4-dihydroisoquinolin-1(2H)-one (63 mg, 0.43 mmol) in DMF (3 mL) cooled at 0° C., NaH (60% suspension in mineral oil, 25 mg, 0.84 mmol) was added and the mixture was stirred at rt for 30 min. The reaction mixture was cooled again at 0° C. and a solution of 2-(trimethylsilyl)ethyl (S)-(3-(4-(chloromethyl)phenoxy)-3-(thiophen-2-yl)propyl)(methy) carbamate (283 mg, 0.64 mmol) and TBAI (16 mg, 0.04 mmol) in DMF (2 mL) was added. The reaction mixture was stirred at rt for 16 h, water was added and extracted with EtOAc. The organic layer was dried with Na₂SO₄, filtered and concentrated under vacuum afford the title compound that was used in the next step without further purification. HPLC (Method B): Ret, 6.51 min; ESI⁺-MS m/z, 573.2 (M+Na).

### b) Title Compound

The compound obtained in step a) was treated with the conditions used in Ex 14 step d) to afford the title compound (70% yield). HPLC (Method A): Ret, 5.91 min; ESI⁺-MS m/z, 407.1 (M+H),

This method was used for the preparation of Ex 51-53 using suitable starting materials:

| | | | | Ret | |
|---|---|---|---|---|---|
| EX | Structure | Chemical name | Method | (min) | MS |
| 51 | 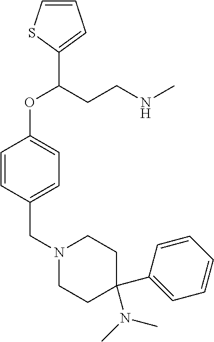 [SMILES: CNCCC(Oc1ccc(CN2CCC(c3ccccc3)(N(C)C)CC2)cc1)c1cccs1] | N,N-dimethyl-1-(4-(3- (methylamino)-1- (thiophen-2- yl)propoxy)benzyl)-4- phenylpiperidin-4- amine | A | 4.14 | 464.3 (M + H) |
| 52 | 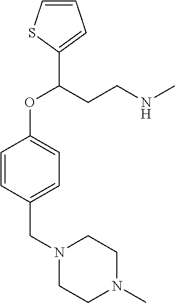 [SMILES: CNCCC(Oc1ccc(CN2CCN(C)CC2)cc1)c1cccs1] | N-methyl-3-(4-((4- methylpiperazin-1- yl)methyl)phenoxy)-3- (thiophen-2-yl)propan- 1-amine | A | 3.79 | 360.2 (M + H) |
| 53 | 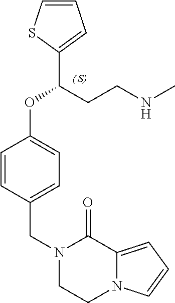 | (S)-2-(4-(3- (methylamino)-1- (thiophen-2- yl)propoxy)benzyl)-3,4- dihydropyrrolo[1,2- a]pyrazin-1(2H)-one | A | 5.31 | 396.1 (M + H) |

Ex 54-55 were prepared by a sequence of reactions according to the methods described in Ex 1 using suitable starting materials:

| | | | | Ret | |
|---|---|---|---|---|---|
| EX | Structure | Chemical name | Method | (min) | MS |
| 54 | 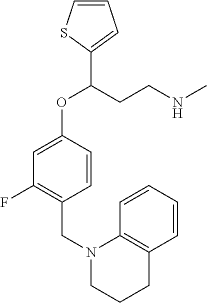 [SMILES: CNCCC(Oc1ccc(CN2CCCc3ccccc32)c(F)c1)c1cccs1] | 3-(4-((3,4- Dihydroquinolin-1(2H)- yl)methyl)-3- fluorophenoxy)-N- methyl-3-(thiphen-2- yl)propan-1-amine | A | 6.95 | 411.2 (M + H) |
| 55 | 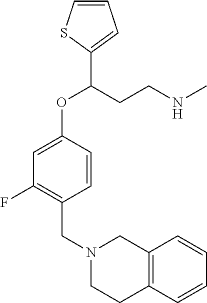 [SMILES: CNCCC(Oc1ccc(CN2CCc3ccccc3C2)c(F)c1)c1cccs1] | 3-(4-((3,4- Dihydroisoquinolin- 2(1H)-yl)methyl)-3- fluorophenoxy)-N- methyl-3-(thiophen-2- yl)propan-1-amine | A | 4.80 | 411.2 (M + H) |

Ex 50 was prepared by a sequence of reactions according to the methods described in Ex 7 using suitahte starting materials:

| | | | | Ret | |
|---|---|---|---|---|---|
| EX | Structure | Chemical name | Method | (min) | MS |
| 56 | 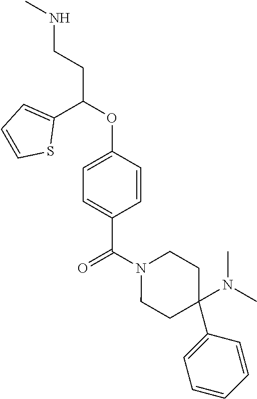 [SMILES: CNCCC(Oc1ccc(C(=O)N2CCC(c3ccccc3)(N(C)C)CC2)cc1)c1cccs1] | (4-(Dimethylamino)-4- phenylpiperidin-1-yl)(4- (3-(methylamino)-1- (thiophen-2- yl)propoxy)phenyl)metha- none | A | 4.79 | 478.2 (M +H) |

Ex 57-58 was prepared by a sequence of reactions according to the methods described in Ex 12 using suitable starting materials:

| | | | | Ret | |
|---|---|---|---|---|---|
| EX | Structure | Chemical name | Method | (min) | MS |
| 57 | 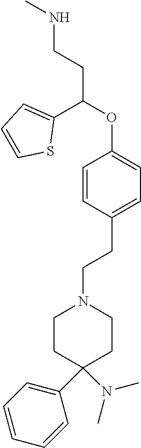 [SMILES: CNCCC(Oc1ccc(CCN2CCC(c3ccccc3)(N(C)C)CC2)cc1)c1cccs1] | N,N-dimethyl-1-(4-(3- (methylamino)-1- (thiophen-2- yl)propoxy)phenethyl)-4- phenylpiperidin-4-amine | A | 4.24 | 478.3 (M + H) |
| 58 | 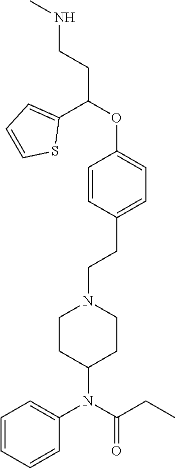 [SMILES: CCC(=O)N(c1ccccc1)C1CCN(CCc2ccc(OC(CCNC)c3cccs3)cc2)CC1] | N-(1-(4-(3- (metyhlamino)-1- (thiophen-2- yl)propoxy)phenethyl)pip- eridin-4-yl)-N- phenylpropionamide | A | 5.14 | 506.3 (M + H) |

### Examples of Biological Activity

### Binding Assay to Human α2δ-1 Subunit of Cav2.2 Calcium Channel.

Human α2δ-1 enriched membranes (2.6 μg) were incubated with 15 nM of radiolabeled [3H]-Gabapentin in assay buffer containing Hepes-KOH 10 mM, pH 7.4.

NSB (non specific binding) was measured by adding 10 μM pregabalin. The binding of the test compound was measured in five different concentrations. After 60 min incubation at 27° C. binding reaction was terminated by filtering through Multiscreen GF/C (Millipore) presoaked in 0.5% polyethyleneimine in Vacuum Manifold Station, followed by 3 washes with ice-cold filtration buffer containing 50 mM Tris-HCl, pH 7.4.

Filter plates were dried at 60° C. for 1 hour and 30 μl of scintillation cocktail were added to each well before radioactivity reading.

Readings were performed in a Trilux 1450 Microbeta radioactive counter (Perkin Elmer).

### Binding Assay Human μ-Oploid Receptor

To investigate binding properties of test compounds to human μ-opioid receptor, transfected CHO-K1 cell membranes (20 μg) were incubated with 1 nM of [³H]-DAMGO in assay buffer containing Tris-HCl 50 mM, MgCl2 5 mM at pH 7.4. NBS (non-specific binding) was measured by adding 10 μM Naloxone. The binding of the test compound was measured at five different concentrations. Plates were incubated at 27° C. for 60 minutes. After the incubation period, the reaction mix was then transferred to MultiScreen HTS, FC plates (Millipore), filtered and plates were washed 3 times with ice-cold 10 mM Tris-HCL (pH 7.4). Filters were dried and counted at approximately 40% efficiency in a MicroBeta scintillation counter (Perkin-Elmer) using EcoScint liquid scintillation cocktail.

The following scale has been adopted for representing the binding to the α2δ-1 receptor expressed as Ki:
+ Ki-α2δ-1>=3000 nM
++ 500 nM<Ki-α2δ-1<3000 nM
+++ 100 nM<Ki-α2δ-1<500 nM
++++ Ki-α2δ-1<100 nM

For the μ-opioid receptor (MOR or mu-opioid receptor) receptor, the following scale has been adopted for representing the binding expressed as KI:
+Kᵢ (μ)>=1000 nM
++ 500<Kᵢ (μ)<1000 nM
+++ 100 nM<Kᵢ(μ)<500 nM
++++ Kᵢ(μ)<100 nM

The results of the binding for the α2δ-1 receptor are shown in Table 1:

TABLE 1

| | Ki (nM) |
|---|---|
| Example | alpha2delta |
| number | Hum |
| 1 | ++ |
| 2 | +++ |
| 3 | +++ |
| 4 | ++ |
| 5 | +++ |
| 6 | ++ |
| 7 | ++ |
| 8 | + |
| 9 | + |
| 10 | + |
| 11 | + |
| 12a | ++ |
| 12b | ++ |
| 13 | + |
| 14 | +++ |
| 15 | ++ |
| 16 | ++ |
| 17 | +++ |
| 18 | + |
| 19 | + |
| 20 | ++ |
| 21 | + |
| 22 | ++ |
| 23 | + |
| 24 | ++ |
| 25 | ++ |
| 26 | ++ |
| 27 | + |
| 28 | ++ |
| 29 | ++ |
| 30 | + |
| 31 | ++ |
| 32 | ++ |
| 33 | + |
| 34 | + |
| 35 | ++ |
| 36 | + |
| 37 | ++ |
| 38 | ++ |
| 39 | ++ |
| 40 | ++ |
| 41 | + |
| 42 | ++ |
| 43 | ++ |
| 44 | +++ |
| 45 | ++ |
| 46 | + |
| 47 | + |
| 48 | ++ |
| 49 | + |
| 50 | ++ |
| 51 | + |
| 52 | + |
| 53 | ++ |
| 54 | + |
| 55 | + |
| 56 | + |
| 57 | + |
| 58 | ++ |

The results of the binding for the α2δ-1 receptor and the μ-opioid receptor (MOR or mu-opioid receptor) for the dual compounds are shown in Table 2:

TABLE 2

| | MU | Ki (nM) |
|---|---|---|
| Example | Ki | alpha2delta |
| Number | nM | Hum |
| 2 | ++++ | +++ |
| 3 | +++ | +++ |
| 4 | +++ | ++ |
| 5 | ++++ | +++ |
| 7 | ++ | ++ |
| 14 | ++++ | +++ |
| 15 | ++++ | ++ |
| 16 | +++ | ++ |
| 17 | ++++ | +++ |
| 20 | +++ | ++ |
| 21 | +++ | + |
| 22 | +++ | ++ |
| 23 | ++++ | + |
| 24 | ++++ | ++ |
| 25 | ++++ | ++ |
| 26 | ++++ | ++ |
| 27 | ++++ | + |
| 32 | +++ | ++ |
| 43 | ++ | ++ |
| 45 | ++++ | ++ |
| 46 | ++++ | + |
| 47 | + | + |
| 51 | +++ | + |
| 54 | ++++ | + |
| 55 | ++++ | + |

1-16. (canceled)

17. A compound of general formula (I): 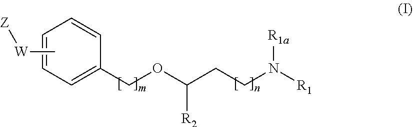
wherein:
R₁ and R₁ₐ are independently from one another a hydrogen atom or a branched or unbranched C₁₋₆ alkyl radical;
R₂ is selected from the group consisting of an optionally substituted 6-membered aryl group and an optionally substituted 5 to 9-membered heteroaryl group having at least one heteroatom selected from the group consisting of N, O and S;
n and m are independently 0.1 or 2;
—W—Z moiety is in meta or para position;
W represents —(CH₂)ₚ— —C(O)— or a bond;
p is 1 or 2;
Z is selected from the group consisting of an optionally substituted 5 to 9 membered heteroaryl group having at least one heteroatom selected from the group consisting of N, O and S; an optionally substituted 3 to 6 membered heterocycloalkyl group having at least one heteroatom selected from the group consisting of N, O and S; and an optionally substituted 5 to 10-membered heterocyclic system having at least one heteroatom selected from the group consisting of N, O and S;
or a pharmaceutically acceptable salt, isomer, prodrug or solvate thereof.

18. The compound according to claim 17, wherein R₂ represents a thiophene or a benzene, which groups are optionally substituted by at least one substituent selected from the group consisting of a halogen atom, a branched or unbranched C₁₋₆-alkyl radical, a branched or unbranched C₁₋₆-alkoxy radical, a C₁₋₆-haloalkoxy radical, a C₁₋₆-haloalkyl radical and a hydroxyl radical.

19. The compound according to claim 17, wherein R₂ represents a group selected from: 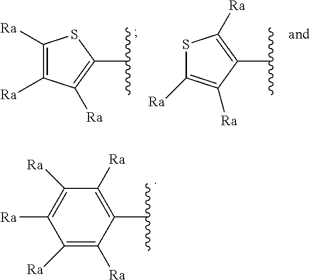
wherein each Rₐ Independently represents a hydrogen atom, a halogen atom, a branched or unbranched C₁₋₆ alkyl radical, a branched or unbranched C₁₋₆-alkoxy radical, a C₁₋₆-haloalkoxy radical, a C₁₋₆-haloalkyl radical or a hydroxyl radical.

20. The compound according to claim 17, wherein Z is selected from the group consisting of: 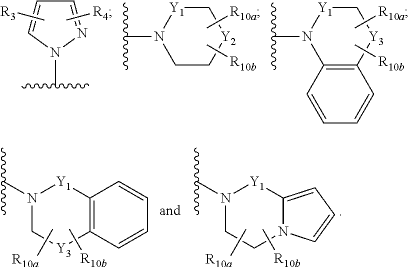
wherein:
R₃ and R₄ are independently from one another a hydrogen atom or a branched or unbranched C₁₋₆ alkyl radical;
Y₁ is —CH₂— or —C(O);
Y₂ is —O—, —NR₅, —CR₆R₇— or the following moiety: 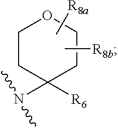
Y₃ is —O—, —NR₅ or —CR₆R₇—;
R₅ is a hydrogen atom or a branched or unbranched C₁₋₆ alkyl radical;
R₆ and R₇ are independently from one another a hydrogen atom, an optionally substituted phenyl radical or a —NR₇ₐR_{7b} radical;
R₇ₐ and R_{7b} are independently from one another a hydrogen atom, a branched or unbranched C₁₋₆ alkyl radical, a phenyl or a —C(O)—C₁₋₆ alkyl radical;
R₈ is a branched or unbranched C₁₋₆ alkyl radical or a —C(O)R₉ radical;
R₈ₐ and R_{8b} are independently from one another a hydrogen atom or a branched or unbranched C₁₋₆ alkyl radical:
R₉ is a an optionally substituted 6-membered aryl radical or a an optionally substituted 5 or 6-membered heteroaryl group having at least one heteroatom selected from the group consisting of N, O and S;
R₁₀ₐ, R_{10b} are independently from one another a hydrogen atom or a branched or unbranched C₁₋₆ alkyl radical;
or R₁₀ₐ or R_{10b}, together with Y₁, Y₂, a carbon atom and the carbon atoms to which they are attached, forms a substituted or unsubstituted aryl or heteroaryl radical having at least one heteroatom selected from the group consisting of N, O and S.

21. The compound according to claim 20, wherein R₃ and R₄ independently from one another represent hydrogen or methyl.

22. The compound according to claim 20, wherein R₉ is an optionally substituted 5 or 6-membered heteroaryl group having at least one nitrogen atom.

23. The compound according to claim 17, wherein R₁ represents a branched or unbranched C₁₋₆ alkyl radical.

24. The compound according to claim 23, wherein R₁ represents methyl.

25. The compound according to claim 17, wherein R₁ₐ represents a hydrogen atom.

26. The compound according to claim 17, wherein R₁ represents a methyl and R₁ₐ represents a hydrogen atom.

27. The compound according to claim 17, which is selected from the group consisting of:
3-(3-((3,5-Dimethylpiperazin-1-yl)methyl)phenoxy)-N-methyl-3-(thiophen-2-yl)propan-1-amine;
N,N-dimethyl-1-(3-(3-(methylamino)-1-(thiophen-2-yl)propoxy)benzyl)-4-phenylpiperidin-4-amine;
3-(1-(3-(3-(methylamino)-1-(thiophen-2-yl)propoxy)benzyl)piperidin-4-yl)phenol;
N-(1-(3-(3-(methylamino)-1-(thiophen-2-yl)propoxy)benzyl)piperidin-4-yl)-N-phenylpropionamide;
3-(4-(Dimethylamino)-1-(3-(3-(methylamino)-1-(thiophen-2-yl)propoxy)benzyl)piperidin-4-yl)phenol;
N-methyl-(3-(piperazin-1-ylmethyl)phenoxy) 3-(thiophen-2-yl)propan-1-amine;
(4-(Dimethylamino)-1-phenylpropoxy-1-yl)(3-(3-(methylamino)-1-(thiophen-2-yl)propoxy)phenyl)methanone;
(3-(3-(Methylamino-1-phenylpropoxy)phenyl)(piperidin-1-yl)methanone;
(3-(3-(Methylamino)-1-phenylpropoxy)phenyl)(4-methylpiperazin-1-yl)methanone;
(3-(3-(Methylamino)-1-phenylpropoxy)phenyl)(morpholino)methanone;
(3-(3-(Methylamino)-1-(thiophen-2-yl)propoxy)phenyl)(4-methyl piperazin-1-yl)methanone;
(R)-3-(3-(2(4-(4-((S)-4-ethyl-2,2-dimethyltetrahydro-2H-pyran-4-yl)piperazin-1-yl)ethyl)phenoxy)-N-methyl-3-phenylpropan-1-amine;
(R)-3-(3-(2-(4-((R)-4-ethyl-2,2-dimethyltetrahydro-2H-pyran-4-yl)piperazin-1-yl)ethyl)phenoxy)-N-methyl-3-phenylpropan-1-amine;
3-(3-(3,5-Dimethyl-1H-pyrazol-1-yl)phenoxy)-N-methyl-3-phenyl propan-1-amine;
(S)-3-(4-(dimethylamino)-1-(3-(3-(methylamino)-1-(thiophen-2-yl) propoxy)benzyl)piperidin-4-yl)phenol;
(S)-N,N-dimethyl-1-(3-(3-(methylamino)-1-(thiophen-2-yl)propoxy)benzyl)-4-phenylpiperidin-4-amine;
(R)-N,N-dimethyl-1-(3-(3-(methylamino)-1-(thiophen-2-yl)propoxy)benzyl)-4-phenylpiperidin-4-amine;
(R)-3-(4-(dimethylamino)-1-(3-(3-(methylamino)-1-(thiophen-2-yl)propoxy)benzyl)piperidin-4-yl)phenol;
((R)-2,2-dimethyl-4-(4-(3-((S)-3-(methylamino)-1-(thiophen-2-yl)propoxy)benzyl)piperazin-1-yl)tetrahydro-2H-pyran-4-yl)(pyridin-2-yl)methanone;
((S)-2,2-dimethyl-4-(4-(3-((S)-3-(methylamino)-(thiophen-2-yl)propoxy)benzyl)piperazin-1-yl)tetrahydro-2H-pyran-4-yl)(pyridin-2-yl)methanone;
N,N-dimethyl-1-(3-((3-(methylamino)-1-(thiophen-2-yl)propoxy)methyl)benzyl)-4-phenylpiperidin-4-amine;
(S)-N,N-dimethyl-1-(3-((3-(methylamino)-1-(thiophen-2-yl)propoxy)methyl)benzyl-4-phenylpiperidin-4-amine;
(R)-N,N-dimethyl-1-(3-((3-(methylamino)-1-(thiophen-2-yl)propoxy)methyl)benzyl)-4-phenylpiperidin-4-amine;
(S)-3-(4-(dimethylamino)-1-(3-((3-(methylamino)-1-(thiophen-2-yl)propoxy)methyl)benzyl)piperidin-4-yl)phenol;
(R)-3-(4-(dimethylamino)-1-(3-((3-(methylamino)-1-(thiophen-2-yl)propoxy)methyl)benzyl)piperidin-4-yl)phenol;
N,N-dimethyl-1-(3-((3-(methylamino)-1-phenylpropoxy)methyl)benzyl)-4-phenylpiperidin-4-amine;
(R)-N,N-dimethyl-1-(3-((3-(methylamino)-1-phenylpropoxy)methyl)benzyl)-4-phenylpiperidin-4-amine;
(S)-N,N-dimethyl-(3-(3(3-(methylamino)-1-phenylpropoxy)methyl)benzyl)-4-phenylpiperidin-4-amine;
((S)-2,2-dimethyl-4-(4-(3-(((R)-3-(methylamino)-1-phenylpropoxy)methyl)benzyl)piperazin-1-yl)tetrahydro-2H-pyran-4-yl)(pyridin-2-yl)methanone;
((R)-2,2-dimethyl-4-(4-(3-(((R)-3-(methylamino)-1-phenylpropoxy)methyl)benzyl)piperazin-1-yl)tetrahydro-2H-pyran-4-yl)(pyridin-2-yl)methanone;
(R)-3-((3-((4-((R)-4-ethyl-2,2-dimethyltetrahydro-2H-pyran-4-yl)piperazin-1-yl)methyl)benzyl)oxy)-N-methyl-3-phenylpropan-1-amine;
(R)-3-((3-((4-((S)-4-ethyl-2,2-dimethyltetrahydro-2H-pyran-4-yl)piperazin-1-yl)methyl)benzyl)oxy)-N-methyl-3-phenylpropan-1-amine;
2-(3-((3-(Methylamino)-1-(thiophen-2-yl)propoxy)methyl)phenyl)-3,4-dihydroisoquinolin-1(2H)-one;
1-(3-(3-((Methylamino)-1-phenylpropoxy)phenyl)piperidin-2-one;
1-(3-(3-(Methylamino)-1-(thiophen-2-yl)propoxy)phenyl)piperidin-2-one;
N-methyl-3-(3-(4-methylpiperazin-1-yl)phenoxy)-3-phenylpropan-1-amine;
4-Methyl-1-(3-(3-(methylamino)-1-(thiophen-2-yl)propoxy)phenyl)piperazin-2-one;
N-methyl-3-(3-(4-methylpiperazin-1-yl)phenoxy)-3-(thiophen-2-yl)propan-1-amine;
1-(3-((3-(Methylamino)-1-phenylpropoxy)methyl)phenyl)piperidin-2-one;
1-(3-((3-(Methylamino)-1-(thiophen-2-yl)propoxy)methyl)phenyl)piperidin-2-one;
N-methyl-3-((3-(4-methylpiperazin-1-yl)benzyl)oxy)-3-phenylpropan-1-amine;
4-Methyl-1-(3-((3-(methylamino)-1-phenylpropoxy)methyl)phenyl)piperazin-2-one;
N-methyl-3-(3-(piperazin-1-yl)phenoxy)-3-(thiophen-2-yl)propan-1-amine;
N,N-dimethyl-1-(3-(3-(methylamino)-1-(thiophen-2-yl)propoxy)phenyl)-4-phenylpiperidin-4-amine;
3-(3-((3S,5R)-3,5-dimethylpiperazin-1-yl)phenoxy)-N-methyl-3-(thiophen-2-yl)propan-1-amine;
N,N-dimethyl-1-(3-((3-(methylamino)-1-(thiophen-2-yl)propoxy)methyl)phenyl)-4-phenylpiperidin-4-amine;
N,N-dimethyl-1-(3-((3-(methylamino)-1-phenylpropoxy)methy)phenyl)-4-phenylpiperidin-4-amine;
3-((3-(3,4-Dihydroquinoxalin-1(2H)-yl)benzyl)oxy)-N-methyl-3-phenylpropan-amine;
N,N-dimethyl-1-(3-((3-(methylamino)-1-phenylpropoxy)methy)phenyl)piperidin-4-amine;
N,N-dimethyl-1-(4-((3-(methylamino)-1-(thiophen-2-yl)propoxy)methyl)phenyl)-4-phenylpiperidin-4-amine;
(S)-2-(4-(3-(Methylami no)-1-(thiophen-2-yl)propoxy)benzyl)-3,4-dihydroisoquinolin-1(2H)-one;
N,N-dimethyl-1-(4-(3-(methylamino)-1-(thiophen-2-yl)propoxy)benzyl)-4-phenylpiperidin-4-amine;
N-methyl-3-(4-((4-methylpiperazin-1-yl)methyl)phenoxy)-3-(thiophen-2-yl)propan-1-amine;
(S)-2-(4-(3-(methylamino)-1-(thiophen-2-yl)propoxy)benzyl)-3,4-dihydropyrrolo[1,2-a]pyrazin-1(2H)-one;
(4-(Dimethylamino)-4-phenyl piperidin-1-yl)(4-(3-(methylamino)-1-(thiophen-2-yl)propoxy)phenyl)methanone;
N,N-dimethyl-1-(4-(3-(methylamino)-1-(thiophen-2-yl)propoxy)phenethyl)-4-phenylpiperidin-4-amine and
N-(1-(4-(3-(methylamino)-1-(thiophen-2-yl)propoxy)phenethyl)piperidin-4-yl)-N-phenylpropionamide;
or a pharmaceutically acceptable salt, isomer, prodrug or solvate thereof.

28. The compound according to claim 17, having one of the following formulas (Id), (Ie), (If), (Ig), (Ih), (Ii) or (Ij): 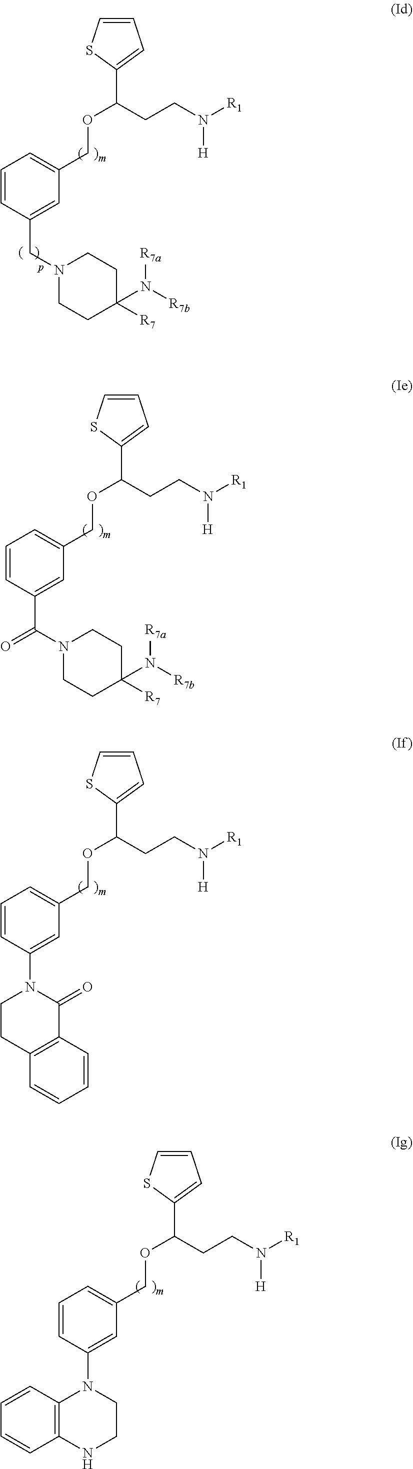 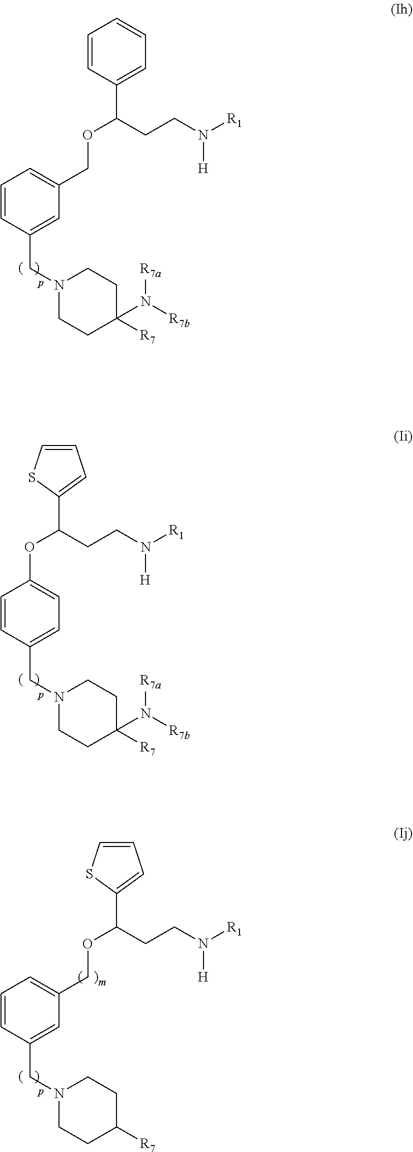
wherein R₁, m and p are as defined in claim 17, and
R₇ is a hydrogen atom, an optionally substituted phenyl radical or a —NR₇ₐR_{7b} radical, wherein R₇ₐ and R_{7b} are independently from one another a hydrogen atom, a branched or unbranched C₁₋₆ alkyl radical, a phenyl or a —C(O)—C₁₋₆ alkyl radical.

29. The compound according to claim 28, which is selected from the group consisting of:
N,N-dimethyl-1-(3-(3-(methylamino)-1-(thiophen-2-yl)propoxy)benzyl)-4-phenylpiperidin-4-amine;
3-(1-(3-(3-(Methylamino)-1-(thiophen-2-yl)propoxy)benzyl)piperidin-4-yl)phenol:
N-(1-(3-(3-(methylamino)-1-(thiophen-2-yl)propoxy)benzyl)piperidin-4-yl)-N-phenylpropionamide;
3-(4-(Dimethylamino)-1-(3-(3-(methylamino)-1-(thiophen-2-yl)propoxy)benzyl)piperidin-4-yl)phenol;
(4-(Dimethylamino)-4-phenylpiperidin-1-yl)(3-(3-(methylamino)-1-(thiophen-2-yl)propoxy)phenyl)methanone;
(S)-3-(4-(dimethylamino)-1-(3-(3-(methylamino)-1-(thiophen-2-yl) propoxy)benzyl)piperidin-4-yl)phenol;
(S)-N,N-dimethyl-1-(3-(3-(methylamino)-1-(thiophen-2-yl)propoxy)benzyl)-4-phenylpiperidin-4-amine;
(R)-N,N-dimethyl-1-(3-(3-(methylamino)-1-(thiophen-2-yl)propoxy)benzyl)-4-phenylpiperidin-4-amine;
(R)-3-(4-(dimethylamino)-1-(3-(3-(methylamino)-1-(thiophen-2-yl)propoxy)benzyl)piperidin-4-yl)phenol;
N,N-dimethyl-1-(3-((3-(methylamino)-1-(thiophen-2-yl)propoxy)methyl)benzyl)-4-phenylpiperidin-4-amine;
(S)-N,N-dimethyl-1-(3-((3-(methylamino)-1-(thiophen-2-yl)propoxy)methyl)benzyl)-4-phenylpiperidin-4-amine;
(R)-N N-dimethyl-1-(3-((3-(methylamino)-1-(thiophen-2-yl)propoxy)methyl)benzyl)-4-phenylpiperidin-4-amine;
(S)-3-(4-(dimethylamino)-1-(3-((3-(methylamino)-1-(thiophen-2-yl)propoxy)methyl)benzyl)piperidin-4-yl)phenol;
(R)-3-(4-(dimethylamino)-1-(3-((3-(methylamino)-1-(thiophen-2-yl)propoxy)methyl)benzyl)piperidin-4-yl)phenol;
N,N-dimethyl-1-(3-((3-(methylamino)-1-phenylpropoxy)methyl)benzyl)-4-phenylpiperidin-4-amine;
(R)-N,N-dimethyl-1-(3-((3-(methylamino)-1-phenylpropoxy)methyl)benzyl)-4-phenylpiperidin-4-amine;
(S)-N,N-dimethyl-1-(3-((3-(methylamino)-1-phenylpropoxy)methyl)benzyl)-4-phenylpiperidin-4-amine;
N,N-dimethyl-1-(3-(3-(methylamino)-1-(thiophen-2-yl)propoxy)phenyl)-4-phenylpiperidin-4-amine;
N,N-dimethyl-1-(3-((3-(methylamino)-1-(thiophen-2-yl)propoxy)methyl)phenyl)-4-phenylpiperidin-4-amine;
N,N-dimethyl-1-(3-((3-(methylamino)-1-phenylpropoxy)methyl)phenyl)-4-phenylpiperidin-4-amine;
3-((3-(3,4-Dihydroquinoxalin-1(2H)-yl)benzyl)oxy)-N-methyl-3-phenylpropan-1-amine and
N,N-dimethyl-1-(4-(3-(methylamino)-1-(thiophen-2-yl)propoxy)benzyl)-4-phenylpiperidin-4-amine;
or a pharmaceutically acceptable salt, isomer, prodrug or solvate thereof.

30. A compound selected from the group consisting of:
3-(4-((3,4-Dihydroquinolin-(2H)-yl)methyl)-3-fluorophenoxy)-N-methyl-3-(thiophen-2-yl)propan-1-amine and
3-(4-((3,4-Dihydroisoquinolin-2(1H)-yl)methyl)-3-fluorophenoxy)-N-methyl-3-(thiophen-2-yl)propan-1-amine;
or a pharmaceutically acceptable salt, stereoisomer or solvate thereof.

31. A process for the preparation of the compound of general formula (I) according to claim 17 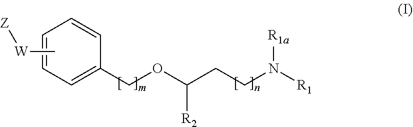
comprising:
A) when m is 0, reaction of a compound of formula (II) 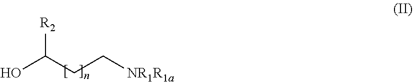
with a compound of formula (IIIa) or (IIIb) 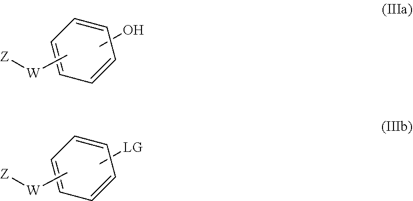
wherein R₁, R₁ₐ, R₂, W, Z and n are as defined in claim 17, and LG represents a leaving group, or
B) when m is 1, reaction of a compound of formula (II) 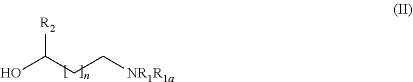
with an alkylating agent of formula (IIIc) 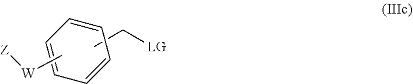
wherein R₁, R₁ₐ, R₂, W, Z and n are as defined in claim 17, and LG represents a leaving group, or
C) starting from an intermediate compound of formula (VIII) 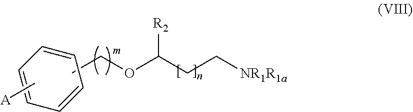
wherein R₁, R₁ₐ, R₂, W, Z, m and n are as defined in claim 17, and wherein A may represent an aldehyde, a carboxylic acid, or a leaving group or (CH₂)ₚ-LG, wherein LG represents a leaving group, and p is 1 or 2, and wherein the reaction is dependent on the nature of A and W resulting in that the reaction comprises:
a reductive amination reaction in the presence of a reductive agent when A is an aldehyde and W is —(CH₂)ₚ—;
reaction in the presence of a carboxilic acid activating reagent when A is a carboxilic acid and WO is a —C(O)— group;
a coupling reaction in the presence of a metal catalyst when A is a leaving group and W is a bond; or
a reaction in the presence of a base when A is —(CH₂)ₚ-LG group and W is a —(CH₂)ₚ— group.

32. A method for the treatment and/or prophylaxis of diseases and/or disorders mediated by the subunit α2δ, especially the α2δ-1 subunit, of voltage-gated calcium channels and/or the μ-opioid receptor (MOR or mu-opioid receptor) in a subject in need thereof, comprising administration of an effective amount of the compound according to claim 17.

33. The method according to claim 32, where the disease or disorder is pain, depression, anxiety and attention-deficit-/hyperactivity disorder (ADHD).

34. The method according to claim 33, wherein the pain is selected from neuropathic pain, inflammatory pain, chronic pain, and other pain conditions involving allodynia and/or hyperalgesia.

35. A pharmaceutical composition comprising a compound according to claim 17, or a pharmaceutically acceptable salt, isomer, prodrug or solvate thereof, and at least a pharmaceutically acceptable carrier, additive, adjuvant or vehicle.
